(19) United States
(12) Patent Application Publication
(10) Pub. No.: US 2007/0037833 A1
(43) Pub. Date: Feb. 15, 2007

(54) Compositions and methods for inhibiting neurodegeneration

(76) Inventors: Robert Gordon Kalb, Philadelphia, PA (US); Jelena Mojsilovic-Petrovic, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/500,772

(22) Filed: Aug. 8, 2006

Related U.S. Application Data
(60) Provisional application No. 60/719,152, filed on Sep. 21, 2005.
(60) Provisional application No. 60/706,278, filed on Aug. 8, 2005.

(51) Int. Cl. A61K 31/522

(52) U.S. Cl. 514/263.33; 514/263.34; 514/263.35

(57) ABSTRACT

Methods effective for inhibiting neuronal degeneration, particularly in ALS patients are disclosed. Also provided are screening assays for identifying such agents.

This application claims priority to U.S. Provisional Application 60/706,278 and 60/719,152 filed Aug. 8, 2005 and Sep. 21, 2005 respectively. Each of the foregoing applications is incorporated herein by reference.

### FIELD OF THE INVENTION

This invention relates to the fields of neurology, signal transduction and pharmacology. More specifically, the invention provides compositions and methods for inhibiting neurodegeneration, particularly in ALS patients.

### BACKGROUND OF THE INVENTION

Several publications and patent documents are cited throughout the specification in order to describe the state of the art to which this invention pertains. Each of these citations is incorporated herein by reference as though set forth in full.

Although the majority of cases of ALS are sporadic, motor neuron disease can also have a genetic etiology (i.e., mutant versions of Cu⁺⁺/Zn⁺⁺ superoxide dismutase 1, the p150^{glued} subunit of dynactin, or Alsin) or result from environmental toxin(s) (Cox et al., 2003; Bruijn et al., 2004). One of the most effective ways to slow the progression of motor neuron death in model systems of ALS is to modulate excitatory glutamatergic neuro-transmission. Neuroprotection is afforded by agents that: 1) block glutamate receptor activation, 2) act pre-synaptically to reduce release of glutamate or 3) augment extracellular glutamate uptake by glial transporters (Rothstein et al., 1993; Estevez et al., 1995; Kwak and Nakamura, 1995; Carriedo et al., 1996; Rothstein et al., 2005). Excitatory neurotransmission is, at the least, a permissive substrate for the toxicity of mutant SOD in vitro and in vivo (Roy et al., 1998; Kruman et al., 1999; Van Damme et al., 2003).

Previous work demonstrated that the vulnerability of motor neurons to excitotoxic insult in vitro is promoted by the peptide growth factor BDNF and is mediated by the receptor tyrosine kinase TrkB (Fryer et al., 2000; Hu and Kalb, 2003). Although this is counter-intuitive, since BDNF-TrkB signaling is essential for survival of populations of developing neurons (Jones et al., 1994; Emfors et al., 1995; Liu et al., 1995; Schwartz et al., 1997; Silos-Santiago et al., 1997), an additional, pro-death activity of BDNF has also been observed in a variety of in vitro, stressed-neuron paradigms (Koh et al., 1995; Ishikawa et al., 2000; Kim et al., 2003). These antipodal actions may be related to the complexity of BDNF-TrkB signaling as well as the maturity of the neurons under study (Kalb, 2005).

Two sets of in vivo observations provide further support for the notion that BDNF might have an adverse action in ALS. First, a human trial of recombinant BDNF treatment for ALS was conducted from February 1998 to December 1999 (BDNF 970278; Investigator-in-Charge, French Centres—Dr. Vincent Meininger (meininge@ccrjussieu.fr)). Patients (total=281) were randomized to receive intrathecal (IT) BDNF 25 μg/day, 150 μg/day or vehicle for 18 months. The primary end point was survival free of ventilatory support. In the two active treatment groups, the survival rate was lower than in the placebo group with a dose effect (worse in the 150 μg/day group than in the 25 μg/day group). Due to the clinical course of subjects in the active treatment groups versus controls, the study was discontinued before the log rank tests reached statistical significance. Second, in a study of the expression of neurotrophin family trophic factors, Kust et al. found a selective increase in the abundance of BDNF message and protein in muscle of ALS patients and this was most pronounced early in the disease (Kust et al., 2002). This collection of in vitro and in vivo observations raise the possibility that pharmacological manipulations that inhibit TrkB signaling might be motor neuron protective.

### SUMMARY OF THE INVENTION

In accordance with the present invention, a method for inhibiting neurodegeneration associated with TrkB signaling is provided. An exemplary method comprises administration of an effective amount of an A2a adenosine receptor antagonist to a patient in need thereof, thereby inhibiting or reducing neuronal damage. The A2a receptor antagonists can optionally be administered with TRK receptor antagonists. Conditions that may be treated using the methods of the invention, include without limitation, ALS, stroke, traumatic injury and epilepsy.

In yet another embodiment of the invention, screening assays for identifying agents which protect neurons from toxic insult are provided. An exemplary method entails providing a population of neuronal cells which are exposed to excitotoxic conditions. The cells are then incubated in the presence and absence of the agent and the amount of cell death determined. Agents which inhibit cell death should have efficacy in the prevention or treatment of conditions associated with neurodegeneration. The cells can also be assessed for other phenotypic and biochemical alterations, including without limitation, disruption in TRK-B signaling, modulation of neuronal cell morphology, disruption in the phosphorylation of TRK-B or SFK, disruption of TRK-B/adenosine A2a receptor interactions and disruption of complex formation in lipid rafts.

### BRIEF DESCRIPTION OF THE DRAWINGS

FIG. 1. Chronic but not acute Adenosine A2a receptor antagonism protects motor neurons from excitotoxic insult in a manner not mimicked by manipulating cAMP levels. Panel a. Immunochemical methods were used to identify motor neurons in mixed spinal cord cultures. Two previously employed markers of motor neurons (Carriedo et al., 1996), non-phosphorylated neurofilament H and peripherin stained a subset of neurons in spinal cord cultures, with large cell bodies and extensive neurites. Virtually all (>98±1%) SMI32 (+) neurons were peripherin (+) and vice versa (merge image). Motor neurons, purified by metrizamide gradient and immunopanning, were live labeled by incubating with CM-DiI and washed motor neurons plated with the remaining population of spinal cord neurons. Immunostaining of cultures revealed that 100% of CM-DiI labeled cells were immunostained with SMI 32(+), anti-choline acetyl transferase and anti-islet ½. CM-DiI labeled cells are seen with conventional rhodamine optics, immunostaining was seen with fluorescein optics and the merged image demonstrates co-localization. Calibration bars in microns (top to bottom)=50, 50, 20 & 10. Panel b. After 14-16 DIV, the excitotoxicity assay was performed: spinal cord neurons were exposed to 100 μM kainic acid (kainate) or vehicle (saline) for 1 hour in Locke's Buffer, washed and 24 hours later the number of CM-DiI labeled cells or cells immunostained with SMI 32, peripherin or ChAT and cell body diameter >20 microns was determined. Kainate treatment caused the loss of ˜45% of motor neurons regardless of the method of cell identification (Student's T-test, *−p<0.01). Panel c. After 14DIV, cultures were exposed to CEP-701, CEP-4416, enprofylline, MRS 1754 or KW6002 for 4 days prior to the excitotoxicity assay. Acute exposure to enprofylline was also studied. Excitotoxic insult lead to a loss of ˜40% of SMI(+) neurons in cultures treated with vehicle treated, MRS 1754 or acute enprofylline treated cultures, while 4 day exposure to CEP-701, CEP-4416, enprofylline or KW6002 completely blocked the loss of SMI(+) neurons (Student's T-test, *−p<0.01). Panel d. DIV14 cultures were exposed to Forskolin/IBMX, SQ 22536, db-cAMP or H89 for 4 days prior to the excitotoxicity assay. No excitotoxic cell death occured in forskolin/IBMX or db-cAMP treated cultures while 40% of SMI(+) neurons were killed in cultures treated with SQ 22536 or H89 (Student's T-test, *−p<0.01).

FIG. 2 Biochemical analysis of the effects of Adenosine or Trk receptor antagonists on cultures of spinal cord neurons. Panel a. 14 DIV cultures of embryonic rat spinal cord neurons grown on astrocyte feeder layer were incubated with MRS 1754 (10 nM), KW6002 (1 μM) or vehicle (DMSO, final concentration 0.1%) for 24 hours and lysates were prepared in RIPA buffer and subjected to immunoblot analysis. KW6002, but not MRS 1754 or vehicle, led to a decrease in the abundance of phosphoTrk and phosphoMAPK without influencing the abundance un-phosphorylated species. Panel b. 14 DIV cultures of embryonic rat spinal cord neurons grown on astrocyte feeder layer were incubated with 100 μM enprofylline, 30 nM CEP-4416 or vehicle (DMSO, final concentration 0.1%) for 6, 24 or 48 hours and lysates were prepared in RIPA buffer and subjected to immunoblot analysis. Enprofylline and CEP-4416 led to a decrease in the abundance of phosphoTrk at all time points with the most profound effect seen at 24 hours. Similarly Enprofylline and CEP-4416 led to a decrease in the abundance of phosphoAKT and phosphoMAPK at 6 and 24 hours with the most profound effect seen at 24 hours. PhosphoMAPK returned to Drug treatment had no effect on the un-phosphorylated species. Panel c. Cultures were prepared as above and immunoblots of cell lysates were probed for phospho-IGFR after treatment with CEP4416 or vehicle for 24 hrs (lane 1 versus 2), or enprofylline or vehicle for 24 hrs (lane 5 versus 6). Neither drug led to an alteration in the abundance in the phosphorylated species. An additional set of cultures were treated for 24 hr with CEP4416 or vehicle (lanes 3 versus 4) or enprofylline or vehicle (lanes 7 versus 8) and then acutely treated with IGF1×30 min. Immunoblots revealed robust induction of phospho-IGFR upon acute stimulation with IGF and neither CEP-4416 nor enprofylline blunted the acute response.

FIG. 3. Co-localization of Adenosine A2a and TrkB receptors on motor neurons and antagonism of either receptor protects against excitotoxic insult. Panel a. Purified motor neurons were live labeled with chick anti-TrkB, washed and fixed prior to incubation with mouse anti-adenosine A2a receptor and species-specific secondary antibodies. All motor neurons were immunostained with both antibodies. The anti-A2a antibody stained an intracellular pool of antigen as well as discrete punta associated with dendrites. The TrkB antibody only stained the cell surface of neurons in both a diffuse and punctate pattern on dendrites. In the merge image several puncta are immunostained for both antigens and inset at higher power shows co-localization of puncta (denoted with arrow heads). Panel b. Lysates of 14DIV spinal cord cultures underwent immune precipitation with anti-Trk or anti-adenosine A2a coated beads and immunoblotted with anti-Trk, anti-adenosine A2a or anti-actin antibodies. Co-immunoprecipation of Trk with adenosine A2a receptors is observed. Actin is not part of this complex. Panel c. Bar graph shows the results from the excitotoxicity assay performed on purified motor neurons pre-treated for 24 hrs with enprofylline, CEP4416 or vehicle. Kainic acid treatment caused the death of approximately 50% of motor neurons in the vehicle treated group (* denotes statistically significantly different, Student's t-test, p<0.01, n=4). No death of motor neurons occurred in the groups pre-treated with enprofylline or CEP4416.

FIG. 4. Time-dependent death of motor neurons triggered by expression of mutant forms of SOD1 and p150^{glued}: neuroprotection afforded by antagonists of Trk, adenosine A2a or AMPA receptors. Panel a. Fourteen DIV spinal cord cultures were infected with recombinant HSV or vehicle (2 μL/ml culture media) and the number of motor neurons determined at two-day intervals thereafter. Repeated measures analysis of variance (RMANOVA) was used to examine the association between the transgene expressed (wild type and mutant forms of SOD and p150^{glued}) and the number of surviving motor neurons over time. For this analysis, the 4 transgene expressing groups, and no-virus control, represent the between-group factor (5 levels) and the survival over the subsequent 8 days (DIV 16, 18, 20 and 22) represents the within-group factor (4 levels). The effects of interest are the group x days interaction and F=13.757, p<0.001 (multivariate analysis, linear model) indicates that survival over time is significantly different as a function of transgene expression. Post hoc comparisons between groups using Scheffé's F test with significance set at p<0.05 showed that the G85R SOD mutant and the G59S p150^{glued} mutant differed significantly from the other groups (denoted with #). No differences in survival were found between the no-virus versus the wt-SOD versus the wt p150^{glued} groups. Panel b. Survival of motor neurons 6 days after infection with HSV expressing the wild type or mutant versions of SOD or p150^{glued}; effects of KW6002. Expression of the mutant versions of SOD or p150^{glued} led to motor neuron death that could be completely reversed by 6 day pre-treatment with KW6002. (Student's T-test, *−p<0.01). Panel c. One set of cultures received function blocking anti-TrkB (Transduction Labs, monoclonal) or a control mouse antibody (ICN Pharmaceuticals, 10A8, anti human apolipoprotein A1) 5 μg/ml final daily×5 days. There were significantly more motor neurons in the anti-TrkB treated cultures compared with the control (denoted with *, Student's t-test, p<0.01, n=6). This experimental paradigm was repeated with HSV G59S p150^{glued} and the same neuroprotective effects seen. Another set of cultures were infected with viruses and received CEP4416 (every other day), enprofylline (every other day), CNQX (daily) or vehicle. There was a significantly greater number of motor neurons in each of the drug treated groups in comparison with the controls both for the HSV-G85R—SOD cultures (F_{7,25}=8.697, p<0.0001) and HSV-G59S-p150^{glued} cultures (F_{7,25}=16.93, p<0.0001). Multiple comparisons were made with ANOVA with Scheffe's post hoc tests with significance set at p<0.05 (denoted by #).

FIG. 5. Relationship between the activation of SFKs and adenosine A2a receptors in Trk activation and neuroprotection. Panel a. shows a motor neuron immunostained for active SFKs (using the clone 28 monoclonal antibody) and adenosine A2a receptors (using a rabbit serum). Immunoreactivity with both antibodies is evident at the cell body and dendrites and much of it co-localizes (yellow in “merge” image). Calibration bar=20 μm. Examination at higher power (images of a single dendrite at right) shows that immuoreactivity is largely puncta and many (but not all) puncta are immuno-positive for both antigens. Calibration bar=7 μm. Panel b. shows immunoblots for active SFKs (clone 28) after 24 hr treatment of spinal cord cultures with MRS 1754, KW6002 or vehicle. The abundance of active SFK is only reduced in cultures treated with KW6002 treatment. Protein loading levels (monitored by actin blots) were equivalent. Panel c. shows immunoblots for active src family kinases (clone 28) after 24 hr treatment of spinal cord cultures with enprofylline or vehicle. The abundance of active SFKs is reduced in the enprofylline treated cultures. Protein loading levels (monitored by actin blots) were equivalent. Panel d. shows the number of motor neurons surviving excitotoxic challenge after treatment with two different methods of inhibiting SFK activation: expression of dominant negative src versus LacZ or treatment with PP1 versus vehicle. Expression of the dominant negative src led to a statistically significant protection when compared with expression of LacZ (* denotes statistically significantly different, Student's t-test, p<0.01, n=5). Similarly, there was a statistically significant greater number of motor neurons after excitotoxic insult in cultures treated with PP1 versus vehicle (Students t-test, p<0.01, n=4). Panel e. shows immunoblots for phosphoTrk after treating cultures for 24 hrs with dominant negative src versus LacZ or PP1 versus vehicle. Both methods for SFK inhibition led to a reduction in the abundance of phosphorylated Trk. Protein loading levels (monitored by actin blots) were equivalent. Panel f shows a motor neuron immunostained for active, phosphoTrk (using a rabbit serum) and active SFKs (using the clone 28 monoclonal antibody). Immunoreactivity with both antibodies is evident at the cell body and dendrites and as in Panel a much of it co-localizes (yellow in “merge” image). Calibration bar=17 μm. Examination at higher power (images of a dendrite at right) shows that immunoreactivity is largely puncta and many (but not all) puncta are immuno-positive for both antigens. Calibration bar=3 μm.

FIG. 6 Trk receptors, adenosine A2a receptors and SFKs are present in lipid rafts and non-lipid rafts. Panel a. Discontinuous sucrose gradients were used to separate rafts from non-lipid rafts and aliquots from 1 ml fractions of the gradient were subjected to western blots for Trk and active SFKs. The lipid raft fraction (“2”) contains all three proteins. Trk and active SFKs are also present in non-raft fractions. Lower panels (left) show co-immunoprecipitation (pull down with anti-Trk beads, blot with anti-adenosine A2a receptor) of A2a with Trk in lipid raft fractions (2) and non-raft fractions (10). The adenosine A2a receptor also associates with P-Trk in both the lipid raft fractions and non-raft fractions (pull down with anti-P-Trk beads, blot with anti-adenosine A2a receptor). Active SFKs can be coIP'ed from rafts and non-raft fractions with anti-A2a or anti-P-Trk (lower right). In the lipid raft fractions, the slower migrating SFK preferentially associates with Trk and adenosine A2a receptors. In the non-lipid raft fractions, the faster migrating SFK preferentially associates with Trk and adenosine A2a receptors. Panel b. Purified motor neurons were grown in the presence of CT-1 and then acutely exposed to BMCD or vehicle followed by BDNF or vehicle. The excitotoxicity assay was performed (exposure kainate or vehicle) and the number of motor neurons assessed one day later. Excitotoxic cell death occurred in motor neurons exposed to BDNF, but did not occur if cells were pre-treated with BMCD prior to BDNF. BMCD itself was non-toxic.

FIG. 7. Model for transactivation of TrkB receptors by adenosine A2a receptors. A complex of adenosine A2a receptors, src non-receptor kinases and Trk receptor kinases is depicted in the lipid raft portion of the plasma membrane. This complex is also known to reside in non-lipid raft membrane. Adenosine A2a receptors have 7 transmembrane domains, src associates with membranes through lipid modification and Trk receptors have a single transmembrane domain. Binding of adenosine (yellow star) to adenosine A2a receptors leads to activation of src, which then phosphorylates Trk receptors. Phosphorylated Trk receptors, through a series of associated proteins (not shown) leads to the activation of downstream signaling molecules such as MAP kinase and PI3'K. Activity of these signaling molecules influences the vulnerability of neurons to toxic insults.

### DETAILED DESCRIPTION OF THE INVENTION

The death of motor neurons in amyotrophic lateral sclerosis (ALS) is thought to result from a variety of factors including excitotoxicity, accumulation of toxic proteins and abnormal retrograde axonal transport. The susceptibility of motor neurons to excitotoxic insults can be limited by inhibiting signals evoked by brain-derived neurotrophic factor (BDNF) activation of the receptor tyrosine kinase TrkB. This can be achieved by direct kinase inhibition, or by blockade of a trans-activation pathway that utilizes adenosine A2a receptors and src-family kinases. Downstream signaling cascades are inhibited by these agents; the phosphoinositide 3′ kinase cassette particularly robustly. In addition to protecting motor neurons from excitotoxic insult, these agents also prevent toxicity that follows from the expression of mutant proteins (G85R,Superoxide Dysmutase; G59S p150^{glued}) that cause familial motor neuron disease.

In addition to the canonical pathway of TrkB activation involving binding of BDNF to the extracellular portion of the receptor, Trk receptors can be trans-activated by intracellular pathways initiated by ligand binding to specific G-protein coupled receptors (Lee et al., 2001; Lee et al., 2002). Activation of adenosine A2a receptors leads to an increase in Trk phosphorylation and src-family kinases which appears to act as intermediaries in this pathway (Lee et al. 2001). Thus, we assessed whether manipulation of adenosine A2a receptor activation influences Trk signaling in spinal cord neurons. We hypothesized that such modulation would provide neuroprotection against excitotoxic injury as well as the baleful actions of mutant proteins previously shown to cause familial forms of motor neuron disease. Thus, in accordance with the present invention compositions and methods are provided which effectively inhibit of Trk signaling, thereby inhibiting or preventing neurodegeneration, particularly in ALS patients.

The protective effect of A2a antagonists has been demonstrated herein in tissue culture models. A2a antagonists may be used therapeutically in patients who suffer from neuronal damage including ALS. While the A2a adenosine receptor antagonist, 3-propylxanthine (enprofylline) is exemplified herein, other antagonists, such as those described in U.S. Pat. No. 5,859,019 to Liang et al. may also be employed.

For therapeutic use, the compounds of the invention may be administered in any conventional dosage form in any conventional manner. Routes of administration include, but are not limited to, intravenous, intramuscular, subcutaneous, intrasynovial, infusion, sublingual, transdermal, oral, topical or inhalation via a nebulizer for example. The preferred modes of administration are via the systemic and inhalation routes.

The compounds of this invention may be administered alone or in combination with adjuvants that enhance stability of the antagonists, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics for the treatment of neurodegeneration (e.g., minocyclin, riluzole) or other adjuvants into a single pharmaceutical composition. Advantageously, the compounds may then be administered together in a single dosage form. In some embodiments, the pharmaceutical compositions comprising such combinations of compounds contain at least about 15%, but more preferably at least about 20%, of a compound of the invention (w/w) or a combination thereof. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 10-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, the patient's age, and the judgment of the treating physician.

Alternatively, a time release or slow release preparation may be utilized which allows for periodic or constant release of the antagonists over a given time period. This method would allow for a single dose of the antagonists in a given day. Methods for preparing such capules are well known to those of skill in the art of drug delivery.

The following materials and methods are provided to facilitate the practice of the present invention. They are not intended to limit the invention in any way.

Sources of Reagents:

CEP4416 and 701 (Cephalon, Inc., Frazer, P A), IGF1 (Bachem), CM-DiI (Molecular Probes), SQ 22536 (Biomol), H89 (CalBiochem) and PP1 (Pfizer). All other chemicals were obtained from Sigma and were of the highest possible grade. The source of tissue culture materials has been described previously (Hu and Kalb, 2003). KW6002 was a gift from Jacques Petzer.

Preparation of Spinal Cord Cultures:

Embryonic Sprague-dawley rat spinal cord neurons were grown on previously established cortical astrocyte monolayers as previously described (Hu and Kalb, 2003). Briefly, the cerebral cortex of postnatal day (P) 1-3 rat pups was dissociated and plated on tissue culture plastic (Primeria) or laminin/polylysine coated glass overslips and maintained in Minimal Essential Media (10% horse serum, 10% fetal bovine serum) and 10 μM AraC was added for 1 day to arrest further proliferation when confluent. Under these conditions, no cortical neurons are present in these cultures. Spinal cord from embryonic (E) day 15 rats was dissociated and plated on the astrocytes at a density of 1 spinal cord/4 ml of media previously conditioned over astrocytes with 10 ng/ml trophic factors added (ciliary neurotrophic factor, cardiotropin 1, BDNF, neurotrophin 4 and glial derived neurotrophic factor, all from Alomone Labs, Israel). Unless otherwise specified, BDNF was added to all cultures. Approximately 50% of media was replaced every third day with fresh media until cultures were used after 14 days in vitro (DIV) for biochemistry or toxicity assays. Purified motor neuron cultures were prepared as previously described using metrizamide gradient and immunopanning techniques (Fryer et al., 1999; Fryer et al., 2000). Dye labeling of motor neurons was accomplished by incubating purified motor neurons with chloromethyl DiI (2 RM) for 5 minutes at 37° C. then 20 minutes at 4° C., washing 3× times in culture media and adding these cells to the dissociated non-motor neuron spinal cord neurons at the time of plating.

In studies employing CEP4416 and 701, the vehicle solution to which neurons was exposed contained dimethyl sulfoxide (DMSO) at a final concentration of 0.2%. A recent study showed that DMSO at concentrations as low as 0.02% can potentiate N-methyl-D-aspartate (NMDA) and non-NMDA excitatory post-synaptic potentials (Tsvyetlynska et al., 2005). To control for any confounding effect of DMSO, all experiments included controls exposed to vehicle with a final DMSO concentration of 0.2%.

Live Labeling of Cell Surface TrkB.

Twenty-fours hours after plating, chicken anti-N-terminal TrkB was added to purified motor neuron cultures with a final dilution of 1:500. After 1 hr. at 37° C., cultures were washed twice in room temperature phosphate buffered saline (PBS), fixed in 4% paraformaldehyde×20 min. and then washed 3× in PBS. Identical results were obtained by incubating with the anti-TrkB antibody at 4° C.

Western Blots and Immunoprecipitation:

Cells were lysed in 1% NP-40 lysis buffers containing protease inhibitors, subjected to SDS-PAGE and immunoblotted as previously described (Fryer et al., 1999; Fryer et al., 2000). For immunoprecipitation, lysates from 60 mm dishes were pre-cleared by incubation with protein A beads (Santa Cruz), incubated with primary antibody (4 μg/ml)×2 hours then 50 μL of beads added and incubated overnight with gentle agitation at 4° C. Beads were pelleted, washed 5× with 10 mM Tris pH 7.4+0.5 mM EGTA+0.5 mM EDTA+0.1% NP40 before elution in 1× Laemelli sample buffer. Antibody sources: BD Transduction Labs—TrkB and MAP Kinase used at 1:500, Cell Signaling—Akt 1:1000; phosphoTrk (tyr490) 1:1000; phospho p42/44 MAP Kinase (thr 202/tyr204) 1:1000; phosphoAkt (ser473) 1:500; IGFI antibody (tyr 1131) 1:500. Upstate—adenosine A2a 1:1000. Sigma—actin 1:200. Biosource—active src (clone 28) 1:200.

Preparation of Lipid Rafts:

We employed previously published methods for the isolation of lipid rafts (Kawabuchi et al., 2000; Suzuki et al., 2004). Briefly, neonatal rat spinal cords (0.25 grams) were homogenized in Triton X-100 Lysis Buffer (50 mM tris HCl pH 8.0, 10 mM MgCl₂, 0.15 mM NaCl, 1% Triton X, 5% glycerol, 20 mM NaF, 1 mM Na₃VO₄, 5 mM β-mercaptoethanol, 10 ug/ml aprotinin, 10 ug/ml leupeptin, 1 mM PMSF) at a 8:1 ratio of buffer to tissue. Two ml of lysate was combined with 2 ml 80% Sucrose in Buffer A, and layered over 5 ml of 30% Sucrose and 1 ml 5% Sucrose (Buffer A: 500 mM NaCl, 10 mM MgCl₂, 50 mM Tris-HCl pH 7.5, 1 mM Na₃VO₄). Using an SW-40 rotor, the samples were spun 12 hrs at 200,000 g. Ten 1 ml samples were removed from the top and labeled 1-10 and used in subsequent analysis.

Excitotoxicity Assays:

The media was removed (and saved), cells were exposed to 100 μM kainic acid or vehicle in Locke's buffer for one hour, washed three times in Locke's buffer not containing kainic acid and the original media restored to each dish (Hu and Kalb, 2003). Twenty-four hours later, dishes were fixed in 4% paraformaldehyde, washed extensively and immuno-stained. Coverslips were incubated with primary antibody overnight, washed and incubated with species-specific secondary antibodies. When double labeling using two spectrally distinct chromophores, control incubations omitting either primary antibody were employed to confirm staining specificity. To quantify the number of stained cells, we counted the number of labeled cells from three non-overlapping fields using a 5× objective on a Zeiss upright Axioskop microscope and the value from one coverslip was averaged. The results from 3-5 independently treated coverslips were the basis of the means and variance used in subsequent statistical analysis. When cell body diameter was assessed in select experiments, a reticule was introduced into an eye piece and the length of the long axis of a neuron noted. Absolute values were made using a micrometer calibration slide (Swift). Antibody sources: Stemberger Monoclonals, Inc —SMI-32 1:1000, Chemicon International ChAT 1:500; Peripherin 1:200; Islet ½ Developmental Studies Hybridoma Bank undiluted 4D5 supernatant. Alexa 488 and 594 conjugated species-specific secondary antibodies were obtained from Molecular Probes and used at 1:200.

Determination of Glutamate Concentration.

Amino acids were derivatized with fluoraldehyde o-phthaldehyde (Pierce) and subjected to liquid chromatography (Varicon 9010).

Determination of Cholesterol Content.

Two hundred thousand motor neurons were plated in wells of a 96 well dish and after 24 hrs. treated with βMCD or vehicle. Thirty minutes later cholesterol content was determined using the Amplex® Red Cholesterol Assay kit (Molecular Probes) according to the manufacturers instructions. All determinations were made in quintuplicate and normalized to protein content (determined using Protein Assay kit (Bio-Rad)).

Determination of BDNF Concentration.

We measured the concentration of BDNF in culture media using ELISA (BDNF Eₘₐₓ immunoassay, Promega) according to the manufacturers instructions.

Recombinant HSV.

Various cDNAs were cloned into the PrpUC amplicon plasmid and were used to generate recombinant HSV as previously described (Neve et al., 1997). The titer of viruses used in these studies were routinely 3-5×10⁷ plaque-forming units/ml. The cDNA for wild type/mutant SOD and wild type/mutant p150^{glued} were gifts of D. Borchelt and E. Holzbaur, respectively. The K295R mutant version of chick src was a gift of J. Brugge.

Cell Imaging.

Images of motor neurons were obtained on an Olympus BX51 microscope equipped with FV300 laser confocal optical microscope utilizing the Fluoview version 4.3 software. Images were obtained with a 40×1.00 N.A. oil UPlan Apo objective at room temperature. Abode® Photoshop CS version 8.0 for MacIntosh Computers was used to crop and assemble images into figures but no alterations in contrast, brightness, hue or gamma setting were undertaken.

Statistical Analysis.

The results from at least 4 independent cell survival or biochemistry experiments are reported here. Every observation within any individual cell survival experiment was obtained in triplicate. Means and standard deviation are reported.

When two groups of observations were compared, Student's t-test was used; when three or more groups of observations were compared, ANOVA was used. Repeated measures analysis of variance (RMANOVA) was used to examine the association between the transgene expressed (wild type and mutant forms of SOD and p150^{glued}) and the number of surviving motor neurons over time.

The following example is provided to illustrate certain embodiments of the invention. It is not intended to limit the invention in any way.

### EXAMPLE I

### Antagonism of Adenosine A2A and Trk Receptors Protects Motor Neurons from Toxic Insult

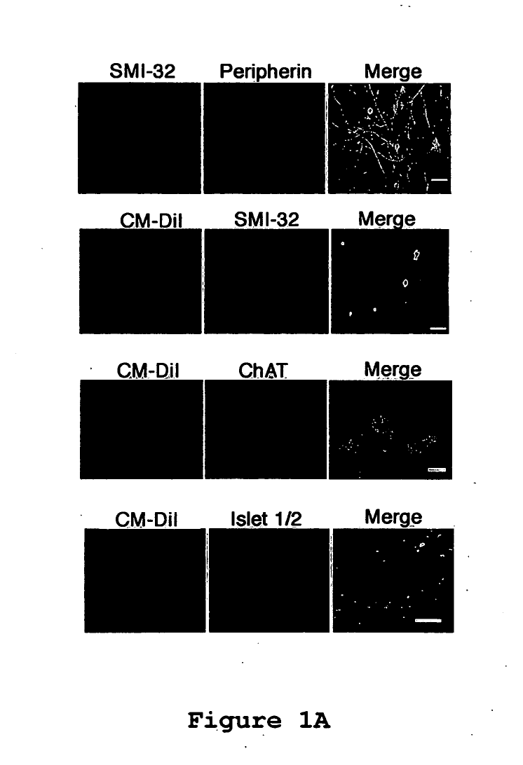

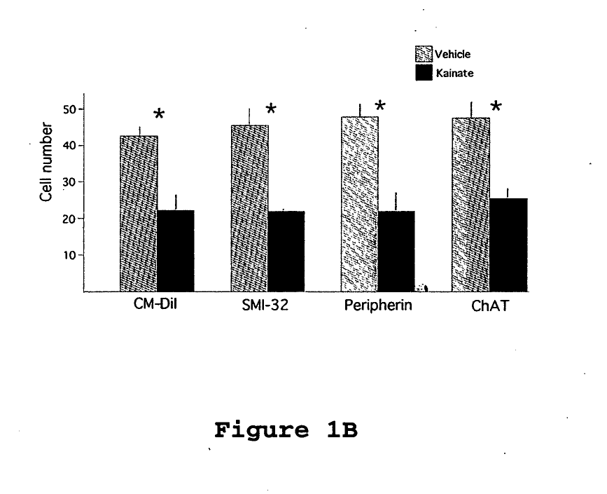

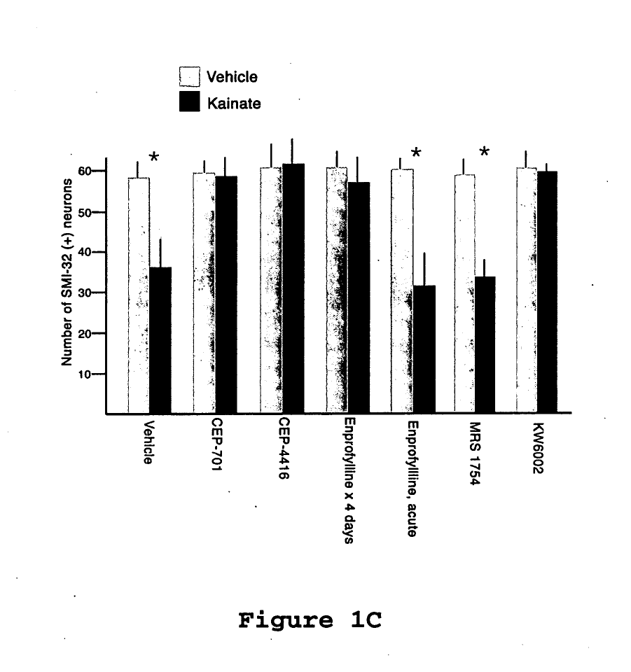

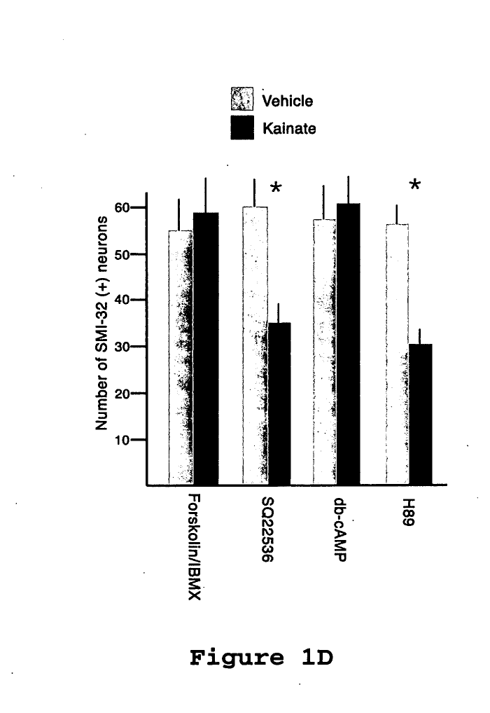

Many previous in vitro investigations have identified motor neurons in mixed spinal cord cell culture by immunostaining for non-phosphorylated neurofilaments (or peripherin) and restricting analysis to cells with a soma diameter of 25 μm or greater (Carriedo et al., 2000; Hu and Kalb, 2003). We wanted to confirm the validity of this approach using an independent method for determining whether a given cell is a motor neuron. To specifically identify motor neurons in our cultures we purified these cells by metrizamide gradient and immunopanning techniques (Henderson et al., 1993; Fryer et al., 1999), live-labeled them with CM-DiI, and added these fluorescently-tagged, bona fide motor neurons to mixed cell cultures at the time of initial plating. All CM-DiI labeled cells stained for motor neurons markers such as non-phosphorylated neurofilament H (using antibody, SMI-32), peripherin, choline acetyl transferase (ChAT) and islet ½ (FIG. 1, panel a.). We then subjected spinal cord cultures to an excitotoxic insult and determined motor neuron survival using multiple methods for identifying motor neurons. Regardless of how we quantify motor neuron number (immunostaining for SMI-32, peripherin, CHAT or CM-DiI), we found that an excitotoxic insult led to the loss of ˜50% of motor neurons (FIG. 1, panel b). Since all SMI-32 (+) cells with cell body diameter of 25 μm or greater were CM-DiI (+) and all CM-DiI (+) cells were SMI-32 (+), for the remainder of the studies herein, we could confidently study motor neuron death in vitro by counting SMI-32 (+) cells with a cell body diameter of 25 μm or greater.

In previous work (employing function blocking anti-TrkB antibodies or by expressing a dominant negative TrkB construct), we showed that antagonism of TrkB activation protected motor neurons from excitotoxic insult (Hu and Kalb, 2003). To be able to translate this work into therapeutics we wanted to identify small molecules that either directly or indirectly inhibit TrkB activation. We examined the neuro-protective efficacy of derivatives of the Trk antagonist K252a that have in vivo activity and have been used in humans (Evans et al., 1999; Evans et al., 2001; Smith et al., 2004). CEP4416 (30 nM), CEP701 (30 nM) or vehicle (0.2% DMSO) was added to spinal cord cultures 4 days and 2 days prior to an excitotoxic insult with Kainic acid (100 μM). No motor neuron death occurred in cultures that were treated with the Trk antagonists while ˜50% of motor neurons were killed in vehicle pre-treated cultures (FIG. 1, panel c.). Thus, direct pharmacological inhibition of Trk activation protects motor neurons from excitotoxic insult.

Stimulation of adenosine A2a receptors causes Trk receptor activation (monitored by receptor phosphorylation and increases in downstream signaling), a process referred to as transactivation (Lee and Chao, 2001; Rajagopal et al., 2004). Since adenosine receptors are typically tonically active in vitro (due to ambient adenosine in the serum of growth media and the efficient generation of adenosine from extracellular ATP by exonucleases (Hirschhorn et al., 1981; Dunwiddie et al., 1997)) we asked whether they were transactivating TrkB in our culture system and contributing to the TrkB-induced vulnerability of motor neurons to excitotoxic insult. One attraction of adenosine receptor antagonists is that they are small molecules in clinical use for human disease (Kase et al., 2003). We began to study this issue by treating spinal cord cultures (grown in the presence of a cocktail of trophic factors including 10 ng/ml BDNF) 4 and 2 days prior to excitotoxic insult with enprofylline. This antagonist of adenosine A1, A2a and A2b receptors (relative potency A2b>A2a>>A1) has good bioavailability, is commercially available and is 10-100 times less expensive than other adenosine receptor antagonists (Robeva et al.). We also studied the selective antagonist of A2a receptors (KW6002, 1 μM) and A2b receptors (MRS1754, 50 nM). There was no excitotoxic motor neuron death in cultures treated with enprofylline or KW6002 and 50% of motor neurons were killed in cultures pre-treated with MRS1754 or vehicle (FIG. 1, panel c.). These results complement our previous observations in which we found that no excitotoxic cell death in cultures pre-treated with ZM241385 (A2a antagonist) but death occurred when cultures were pre-treated with DPCPX (A1 antagonist) (Mojsilovic-Petrovic et al., 2005). Thus 3 distinct compounds that antagonize adenosine A2a receptor were neuroprotective in this experimental paradigm. For most of the subsequent experiments we used enprofylline and KW6002.

The mechanism by which adenosine A2a receptors transactivate Trk receptors is not understood at a cell biological level. One possibility is that active adenosine A2a receptors stimulate the release of BDNF from cells, which then binds to and activates TrkB. The transactivation of vascular smooth muscle cell EGF receptors by angiotensin II has been shown to operate in such an autocrine/paracrine loop (Eguchi et al., 2001). To see if a similar phenomenon was occurring in our cultures, we measured the level of BDNF in the medium of cultures (not provided with exogenous BDNF) treated for two days with enprofylline or vehicle and found no statistically significant drug effect (enprofylline, 33±1.2 pg/ml versus vehicle, 34±4.1 pg/ml, n=6; 2 separate experiments; Students T-test p=0.724). These results argue against the notion that adenosine A2a receptor antagonism modulates TrkB signaling by diminishing the ambient levels of BDNF in our culture system.

Adenosine A2a receptors are known to have rapid modulatory actions on neurotransmission (Dunwiddie and Masino, 2001; Fredholm et al., 2001), raising the possibility that the neuroprotective action of enprofylline is mediated by influencing excitatory neurotransmission among neurons in our cultures. If so, brief pre-treatment of cultures with enprofylline might also be neuroprotective. To test this we grew cultures as described above and added enprofylline to the media 30 minutes prior to the excitotoxic challenge (FIG. 1, panel c.). We counted the number of motor neurons 24 hours later and found that in this paradigm, enprofylline was not neuroprotective (% motor neuron death in short term enprofylline group −46±4 versus % motor neuron death in short term vehicle group −43±5; t-test, p>0.40). In addition, analysis of ambient glutamate concentrations in the media of cultures treated with enprofylline or vehicle for 24 hrs. also revealed no differences between the groups (2.2±0.7 μM versus 1.7±0.4 μM; t-test, p=0.47). These findings argue against the idea that the neuroprotection afforded by A2a antagonism is mediated by rapid alteration of excitability by the drug or changes in extracellular concentration of glutamate.

Transactivation of some receptor tyrosine kinases by GPCRs occurs in a G-protein dependent manner (Oak et al., 2001) and thus we wanted to examine this possibility in our system. Adenosine A2a receptors are GPCRs that upon ligand binding lead to Gα loading with GTP, dissociation from Gβγ and the activation of adenylate cyclase (Dunwiddie and Masino, 2001; Fredholm et al., 2001). Adenosine A2a receptor antagonists would be expected to reduce adenylate cyclase activation and result in a reduction in the intracellular level of cAMP. There is plausibility to the idea that reductions in cAMP levels might be neuroprotective since stress resistance in nondividing yeast Saccharomyces cerevisiae is enhanced by reductions in the CYR1 gene, an adenylate cyclase (Fabrizio et al., 2001). If neuroprotection was mediated by a reduction in cAMP levels, then agents that reduce cAMP levels (such as the adenylate cyclase inhibitor SQ 22536) or reduce the activity of cAMP-dependent kinase PKA, (using H89) ought to mimic the neuroprotective actions of adenosine A2a receptor antagonism. Pre-treatment of cultures with the adenylate cyclase inhibitor SQ 22536 (10 μM) for 4 days did not adversely affect the basal survival of motor neurons and did not protect against excitotoxic challenge (42±4% cell death, FIG. 1, panel d.). Similarly treating cultures with H89 (2 μM) did not protect against excitotoxic challenge (53±5% cell death, FIG. 1, panel d.). These results argue that the neuroprotective activity of adenosine A2a receptor antagonism is not mimicked by maneuvers that reduce cAMP levels or downstream signaling via PKA.

For two reasons we wanted to study the effects of raising cAMP levels. First, it would allow us to ask if the neuroprotection afforded by adenosine A2a receptor antagonism is tied to its ability to reduce cAMP levels. If so, maneuvers that raise cAMP levels (i.e., IBMX+forskolin) ought to erase the neuroprotective activity of adenosine A2a receptor antagonism. Second, at higher concentrations enprofylline (but not KW6002) has phosphodiesterase inhibitory activity and thus (at least theoretically) could lead to a rise in cAMP levels. Would elevation of cAMP levels be neuroprotective? Four day pre-treatment of cultures with IBMX (100 μM)+forskolin (10 μM) had no adverse effect on motor neuron survival and this pre-treatment itself protected motor neurons from excitotoxic insult (−3±4% cell death, FIG. 1, panel d.). Similarly, treating cultures with the cell-permeable, non-hydrolyzable cAMP analogue, dibutryl-cAMP (db-cAMP) at 1 mM, 4 and 2 days prior to the excitotoxic insult, was neuroprotective (−2±3% cell death, FIG. 1, panel d.). These findings are consistent with previous work demonstrating that manipulations that raise cAMP levels strongly support the longterm survival of motor neurons (Hanson et al., 1998). Because IBMX+forskolin and db-cAMP were neuroprotective we could not investigate the potential interaction of adenosine A2a antagonists with elevated cAMP levels. In addition we could not dissociate elevation of cAMP levels from the neuroprotective action of enproylline and thus exclude its potential inhibitory action as a phosphodiesterase antagonist. In sum, the neuroprotective activity of adenosine A2a receptor block is at least partially dissociable from an effect on cAMP levels suggesting that it occurs in a G-protein-independent process. We can not rule out the possibility that the neuroprotective activity of enprofylline (specifically) is mediated, in part, by increases in cAMP levels. These findings are consistent with the view, however, that blockade of adenosine A2a receptors protect neurons from insult by virtue of another biochemical activity(ies).

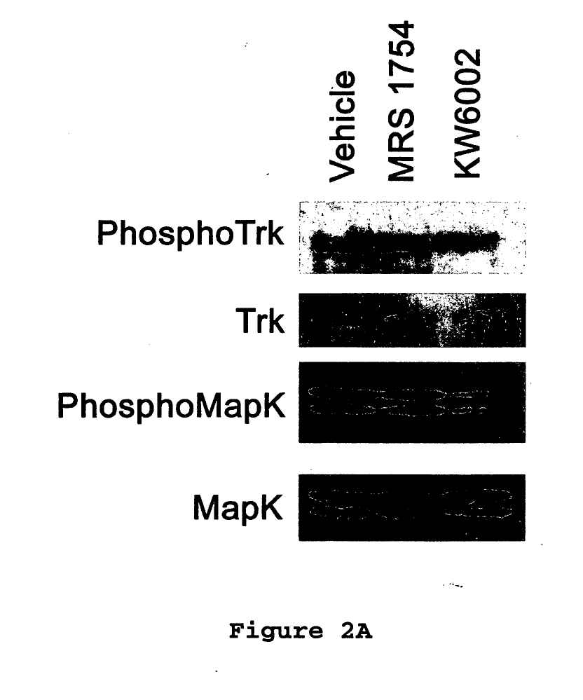

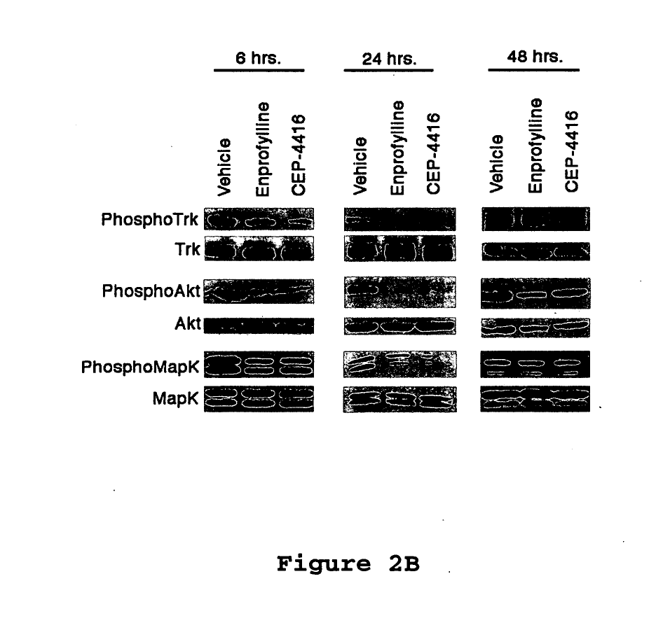

We next asked about the effect of the adenosine A2 receptor antagonism on Trk signaling. Cultures of spinal cord cells (grown in the presence of a cocktail of trophic factors including 10 ng/ml BDNF) received KW6002 (1 μM), MRS1754 (50 nM) or vehicle (DMSO 0.1%) and 24 hrs. later, lysates were prepared for immunoblots for Trk and the MAP kinase downstream signaling module (FIG. 2, panel a). Immunoblots for phosphoTrk and phosphoMAPK were employed to monitor the state of activation of these proteins. KW6002 led to a substantial reduction in the abundance of phosphoTrk and phosphoMAPK without altering the abundance of the unphosphorylated species. The effects of MRS1754 were indistinguishable from the vehicle control. These results indicate that antagonism of adenosine A2a, but not A2b, receptors, inhibits activation of Trk and downstream signaling, even when cells are provided with enough extracellular BDNF to ordinarily activate its receptor.

We followed up these observations by determining the effect of enprofylline on Trk signaling and determined the extent to which the resultant biochemical changes mimic the effects of direct pharmacological Trk antagonism with CEP4416. Six hours post drug administration we regularly detected decreases in the phosphorylation of Trk, MAP kinase and Akt (FIG. 2, panel b.). By 24 hours post drug administration, there was a robust reduction in phosphoTrk, phosphoMAPK and phosphoAKT without any alteration in the overall abundance of the unphosphorylated forms of these proteins. The effect of drug treatment on AKT was particularly marked. 48 hours post drug administration, a decrease in phosphoTrk could still be detected in the drug-treated cells although it was less pronounced than that seen 24 hours earlier. The abundance of phosphoAKT remained depressed and this was particularly evident in the enprofylline treated cells; phosphorylation of MAPK had largely returned to baseline by this time point. To determine the specificity of these agents, we examined their effect on Insulin-like growth factor 1 (IGF1) signaling through the IGF receptor tyrosine kinase (IGFR). Twenty-four hour treatment of spinal cord cultures with CEP4416 or enprofylline had no effect on the basal state of IGFR phosphorylation (FIG. 2, panel c. lanes 1 versus 2; lanes 5 versus 6). In addition, the acute administration of IGF1 (5 nM) to spinal cord cultures led to a strong increase in IGFR phosphorylation and this was undiminished by 24 hour pretreatment with CEP4416 or enprofylline (FIG. 2, panel c. lanes 3 versus 4; lanes 7 versus 8). In sum, both enprofylline and CEP4416 lead to similar reductions in the activation of Trk and the actions of these drugs appears specific, to the extent that they do not interfere with the activation of the IGF1 receptor. The inhibition of signaling downstream of Trk was most pronounced 24 hours after treatment and the blockade of adenosine A2a receptors appears to have a longer lasting effect on the P13'K-AKT pathway. It is unlikely that phosphodiesterase activity of enprofylline can explain these observations as increased cAMP levels enhance, not suppress, TrkB activation (Boulanger and Poo, 1999; Ji et al., 2005).

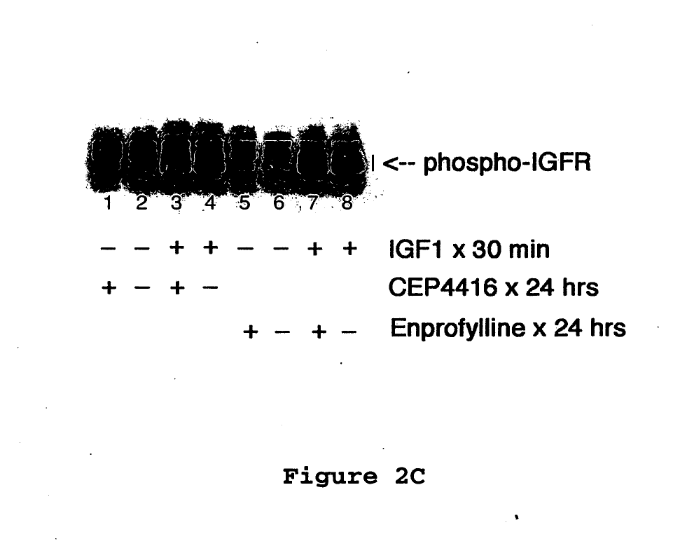

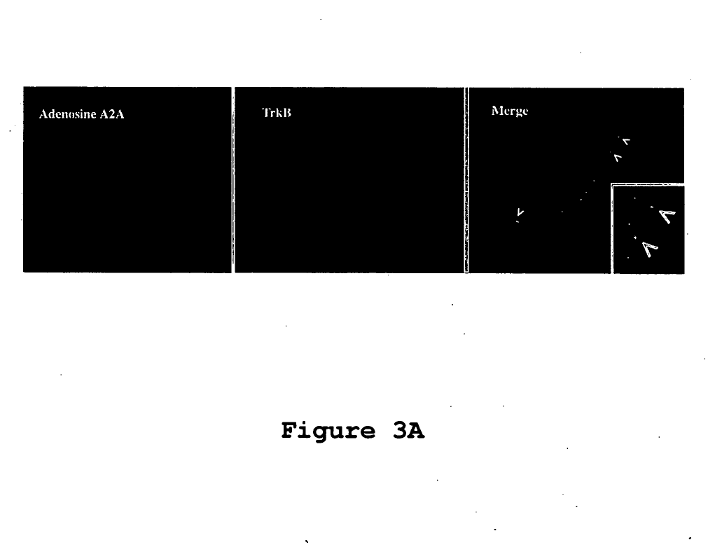

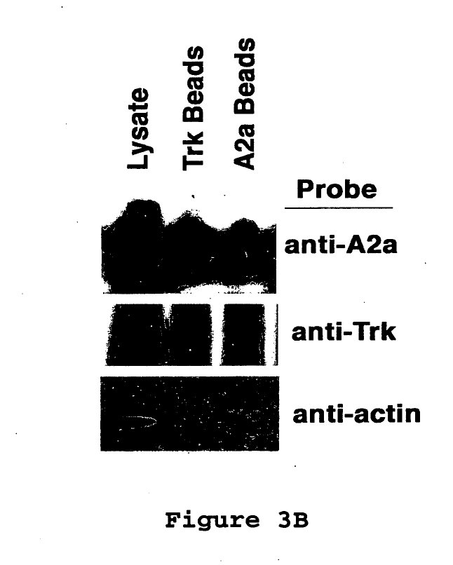

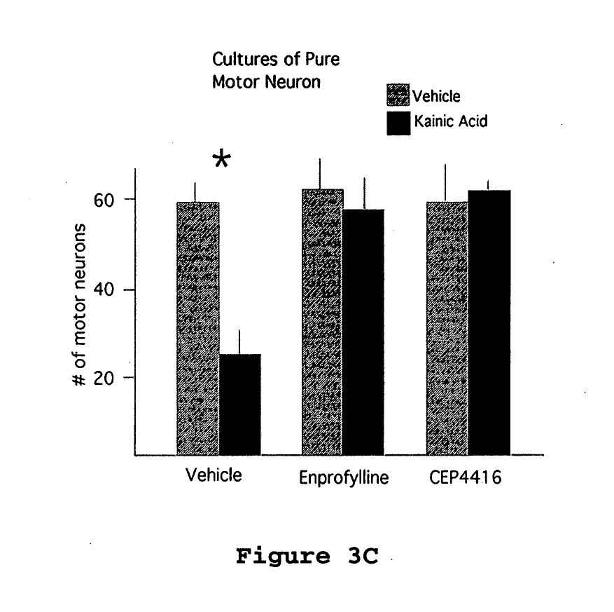

The simplest formulation posits that the modulation of Trk signaling by adenosine receptors occurs on (or within) motor neurons themselves. This is supported by two lines of evidence. First, immunostaining of purified motor neurons demonstrated that all motor neurons are co-labeled with antibodies to Trk and the A2a receptor. In addition to cell body staining, co-localized puncta of Trk and A2a receptor immunoreactivity are evidence on dendritic shafts. The same observation was made on mixed spinal cord cultures where we found that all SMI-32 positive motor neurons were immunoreactive for Trk and A2a (FIG. 3, panel a.). These studies were performed using an antibody to the extracellular, N-terminus of the protein in a live labeling protocol and thus reflect the expression of cell surface TrkB with adenosine A2a receptors. We complemented these observations by subjecting lysates from spinal cord cultures to co-immunoprecipitation analysis. Anti-Trk coated beads immunoprecipitated both Trk and adenosine A2a receptors and conversely anti-adenosine A2a coated beads immunoprecipitated both Trk and adenosine A2a receptors (FIG. 3, panel b.). Beads not coated with primary antibody immunoprecipitated neither Trk nor adenosine A2a receptors. Similar results were obtained with homogenates of spinal cord tissue (not shown). Thus, adenosine A2a and TrkB receptors appear to be components of a macromolecular complex at least a portion of which is on the plasma membrane. Second, we asked if adenosine A2a antagonists (or CEP4416) protected cultures of purified motor neurons from excitotoxic injury. One day after isolation, purified motor neurons grown in the presence of BDNF+CT1 were incubated with enprofylline, CEP4416 or vehicle for 24 hours and then under went excitotoxic challenge (FIG. 3, panel c.). Quantification of motor neuron numbers in the various groups revealed that both drugs had no adverse effects on basal survival. While the excitotoxic insult caused the death of 65±4% of vehicle-treated motor neurons, there was no motor neuron death in the cultures pretreated with Enprofylline (3±1%) or CEP4416 (−2±1%). Thus while a non cell-autonomous interaction between Trk and A2a receptors remains possible, our findings are consistent with adenosine A2a receptor modulation of Trk function at the level of the motor neuron itself.

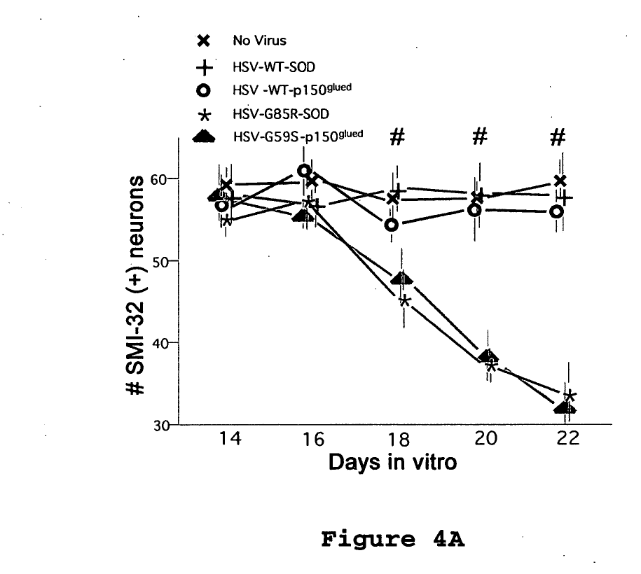

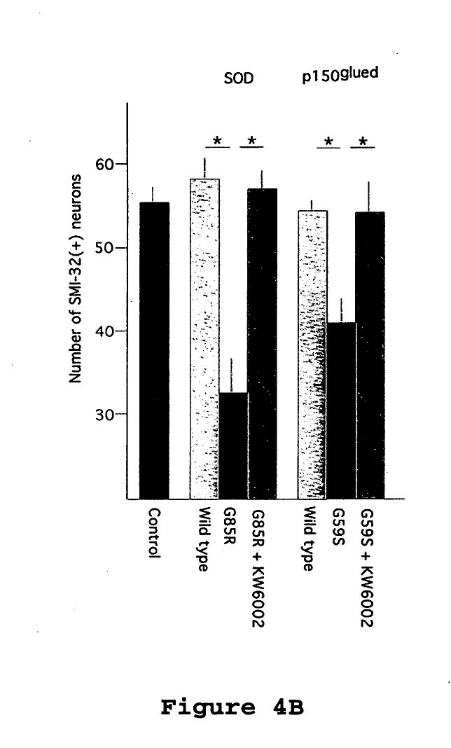

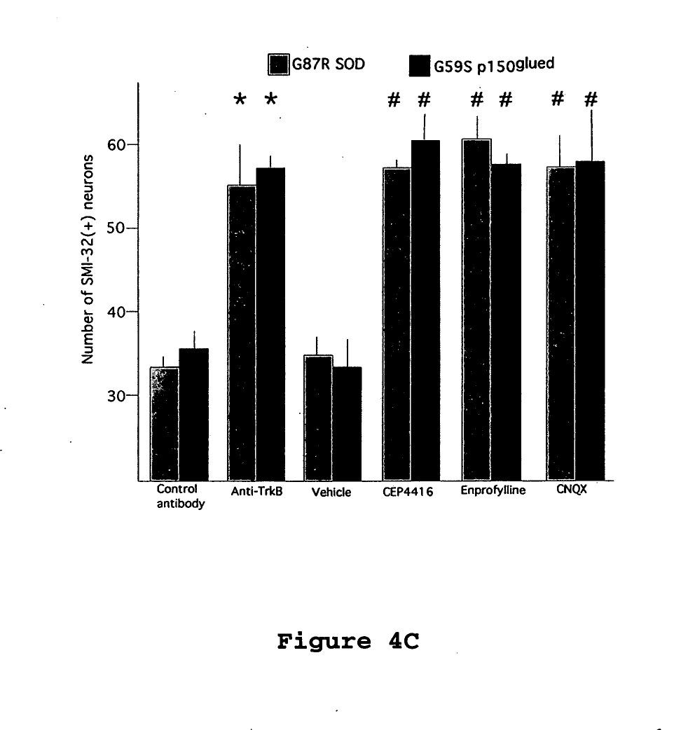

Mutations in superoxide dismutase (SOD) or the p150^{glued} subunit of dynactin are known to account for a familial form of ALS in a subpopulation of individuals (Rosen, 1993; Puls et al., 2003; Bruijn et al., 2004). We inquired whether expression the mutant forms of SOD or p150^{glued} caused motor neuron death in vitro and if Trk antagonism was neuroprotective. Recombinant herpes simplex viruses (HSVs) were generated that expressed wild type or mutant form of SOD (G85R) and wild type or mutant p150^{glued} (G59S). Viruses engineered to express wild type or mutant proteins expressed transgenes at similar levels when assayed by western blot (not shown). Spinal cord cultures were infected with virus at 14 DIV and the number of motor neurons was determined 2, 4, 6 or 8 days later (FIG. 4, panel a.). No decrement in motor neuron number occurred over 8 days in cultures uninfected with virus, or infected with HSV-LacZ, HSV-WT-SOD or HSV-WT-p150^{glued}. In contrast, infecting cultures with HSV-G85R—SOD or HSV-G59S-p150^{glued} led to a progressive loss of motor neurons that differed in a statistically significant manner when compared with controls 4, 6 and 8 days post infection (F=13.757, p<0.001 (multivariate analysis, linear model) repeated measure ANOVA, followed by post hoc Scheffe's test with significance set at p<0.05).

We next determined if KW6002 could protect motor neurons from the toxic effects of mutant proteins. Spinal cord cultures were infected with viruses expressing wild type or mutant versions of SOD or p150^{glued} at 14 DIV, then treated with drug or vehicle for 4 days (FIG. 4, panel b.). The number of motor neurons in cultures uninfected with virus (“control”) or wild type versions of SOD or p150^{glued} were not significantly different (55±2 versus 58±3 versus 54±4). Expression of the mutant versions of SOD or p150^{glued} led to the loss of approximately 40% of motor neurons and KW6002 completely prevented this (F_{6,14}=67.10, p<0.0001, ANOVA). Post hoc analysis (with significant set at P<0.05) showed that the number of motor neurons in the mutant protein expressing cultures was significantly less than motor neurons expressing wild type proteins. KW6002 led to a significant abrogation of the toxic effect of the mutant proteins. These results indicate that in vitro, KW6002 inhibits motor neuron death caused by the expression of mutant versions of SOD or p150^{glued}.

Under normal growth conditions, BDNF is endogenously produced in spinal cord cultures and introduction of function blocking anti-TrkB antibodies daily for 4 days will reduce the abundance of phosphorylated Trk (Hu and Kalb, 2003). To determine if motor neuron death due to G85R—SOD or G59S-p150^{glued} required intact BDNF-TrkB signaling, cultures were infected with the respective viral vectors and were subsequently treated (daily) with function blocking anti-TrkB antibodies or a control antibody (FIG. 4, panel c.). Of note, the neurotrophic factor CT1 was included in all incubations. When we assessed motor neuron survival 6 days post-infection, we found that function blocking anti-TrkB antibodies, but not the control antibody, eliminated the toxicity of the mutant protein (# of motor neurons: 56±2 versus 28±4, anti-TrkB versus control antibody, G85R—SOD1 groups, p<0.01, t-test; 58±2 versus 32±5, anti-TrkB versus control antibody, G59S-p150^{glued} groups, p<0.01, t-test). Simply treating naïve cultures with the function blocking anti-TrkB antibodies had no effect on motor neuron survival. We next asked if enprofylline or CEP4416 (administered every other day for 6 days) or CNQX, an AMPA receptor antagonist, affected the survival of motor neurons in cultures infected with HSV-G85R—SOD or HSV-G59S-p150^{glued} and found that all 3 drugs completely prevented the toxicity of mutant protein expression (FIG. 4, panel c.). All three drug treatments led to a statistically significant neuroprotective effect against the toxic action of G85R SOD (F_{7,25}=8.697, p<0.0001, ANOVA). Similarly all drug treatments led to a statistically significant neuroprotective effect against the toxic action of G59S p150^{glued} (F_{7,25}=16.930, p<0.0001, ANOVA). These results indicate that: 1) antagonism of Trk signaling with function-blocking antibodies or CEP4416 protects motor neurons from the toxicity associated with mutant proteins known to underlie familial ALS, 2) antagonism of adenosine A2a receptor displays similar neuroprotective activity, and 3) basal excitatory neurotransmission ongoing in our culture system provides a necessary substrate for mutant protein toxicity of motor neurons.

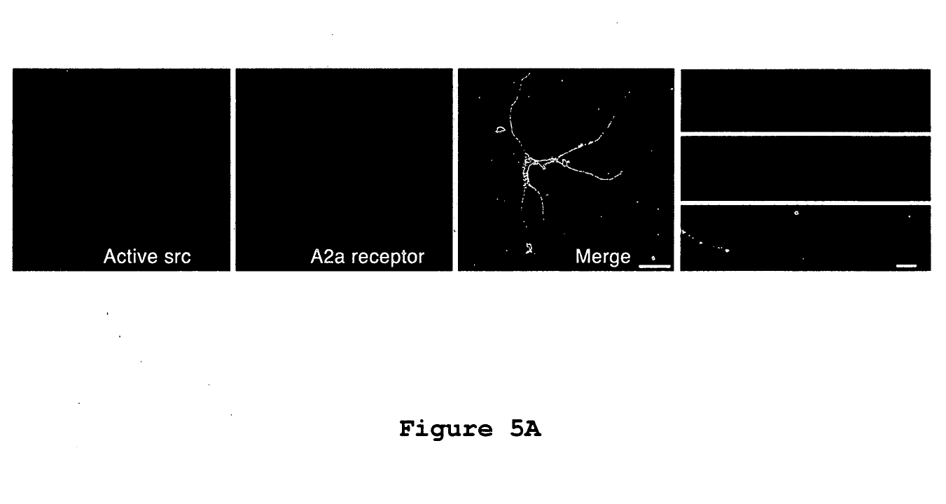

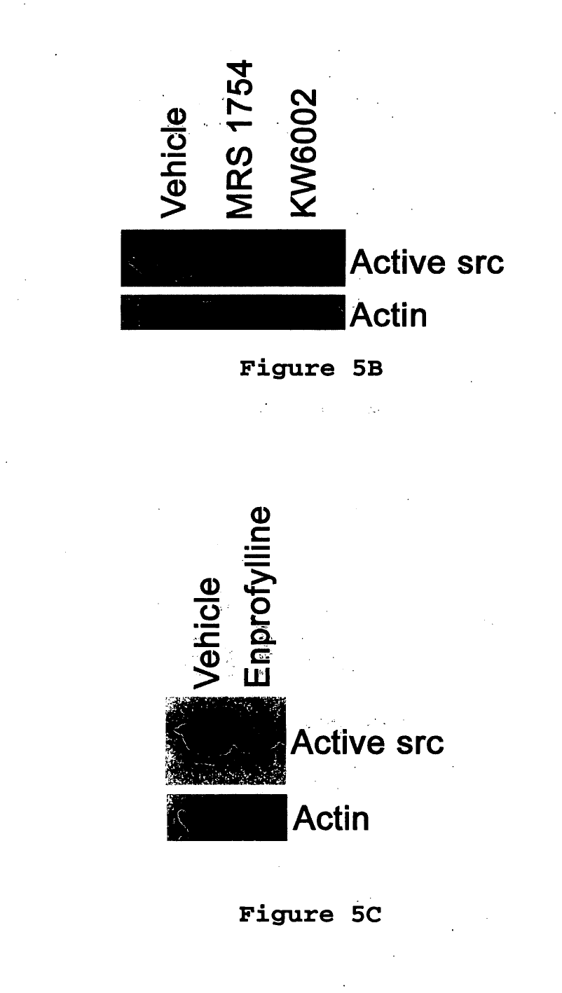

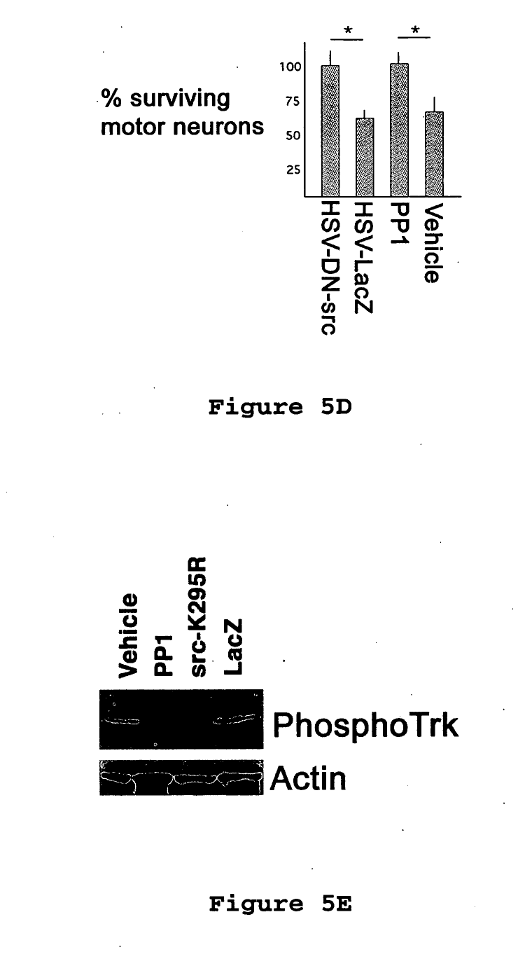

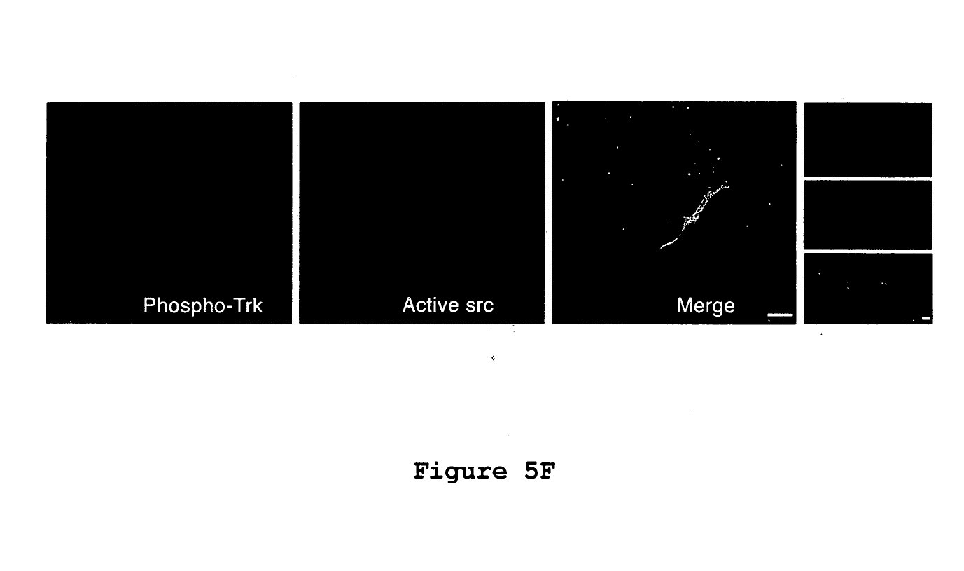

The precise mechanism by which G-protein coupled receptors trans-activate receptor tyrosine kinases is complex and to some extent receptor-subtype specific (Luttrell et al., 1999; Downward, 2003; Piiper and Zeuzem, 2004; Waters et al., 2004). Some evidence indicates that src-family kinases (SFK) participate in adenosine A2a receptors transactivation of Trk (Lee and Chao, 2001). If this pathway was operative in spinal cord neurons, then antagonism of adenosine A2a receptors might lead to reduced src activation and antagonism of SFKs might be neuro-protective. Src is negatively regulated by phosphorylation of tyrosine 529 (refers to human src sequence) and an antibody that recognizes a de-phospho epitope in this region (“clone 28”) is a reliable measure of active src and SFKs (Kawakatsu et al., 1996). We began by asking for adenosine A2a receptors and active SFKs are present in the same cells and if so, their subcellular distribution. Pure motor neurons, grown in the presence of BDNF were immunostained with a rabbit anti-adenosine A2a receptor and the clone 28 antibody. One hundred percent of motor neurons expressed both antigens and on dendrites, the immunoreactivity formed discrete fine puncta (FIG. 5a). Many, but not all, puncta were stained with both antibodies. To study the participation of SFKs in A2a antagonism action, cultures were exposed to KW6002, MRS 1754 or vehicle for 24 hours and lysates probed with the clone 28 antibody. KW6002, but not MRS 1754, caused a reduction in the abundance of active SFKs when compared with vehicle treated cultures (FIG. 5b). We also exposed cultures to enprofylline or vehicle for 24 hours and probed lysates with the clone 28 antibody. Enprofylline too caused a reduction in the abundance of active SFKs when compared with vehicle treated cultures (FIG. 5, panel c). Pharmacological block of SFKs can be achieved with PP1 (Hanke et al., 1996) and we found that pre-treatment of spinal cord cultures with PP1 prevented motor neuron death induced by excitotoxic challenge (% motor neuron survival, 100±2 versus 62±3, PP1 versus vehicle, p<0.01, t-test, FIG. 5c). We employed a second method for inhibiting SFK activity by engineering HSV to express a dominant negative form of src (K295R) in neurons (Mukhopadhyay et al., 1995). Spinal cord cultures were infected with the HSV-K295R-src or HSV-LacZ and 24 hours later the excitotoxicity assay was performed. Expression of the dominant negative src (but not β-galactosidase) rescued motor neurons from the excitotoxic challenge (% motor neuron survival, 99±3 versus 59±2, src-K295R versus LacZ, p<0.01, t-test, FIG. 5c). These experiments reveal that: 1) some adenosine A2a receptors reside in close proximity to active SFKs, 2) SFK activation is downstream of adenosine A2a receptors and 3) the neuroprotective action of adenosine A2a receptor antagonists utilizes a SFK pathway.

We next inquired about the relationship between SFKs and Trk receptors. When pure motor neurons, grown in the presence of BDNF, were immunostained with a rabbit anti-phosphoTrk receptor antibody and the clone 28 antibody we found that one hundred percent of motor neurons expressed both antigens at the cell body and on dendrites (FIG. 5, panel f.). As noted above for adenosine A2a and active SFKs, the immunoreactivity formed discrete fine puncta and in many instances there was co-localization. To determine if this close spatial relationship between active Trk receptors and active SFKs reflected a functional relationship, we monitored the state of phosphorylation of Trk after blocking SFK activity with PP1 or HSV-src-K295R. We found that antagonism of SFK activation led to a reduction in the abundance of phosphoTrk (FIG. 5, panel e.). These findings suggest that active adenosine A2a receptors contribute to SFK activation, and src (or src-family) kinases are upstream of TrkB activation.

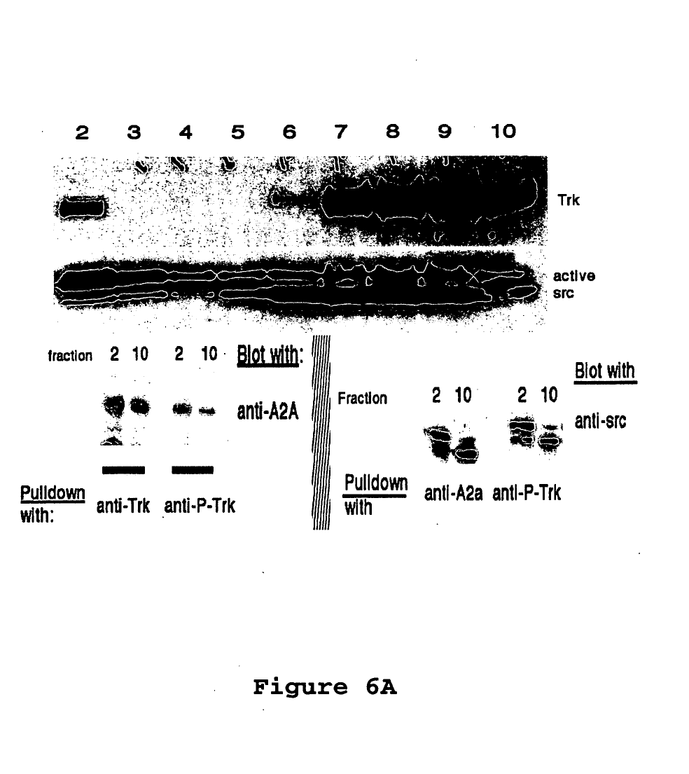

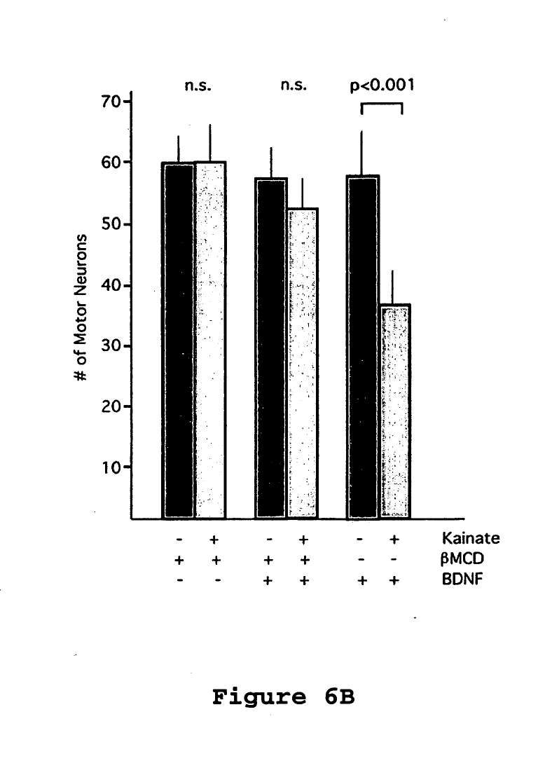

Lipid rafts are cholesterol/sphingolipid rich microdomains of plasma membrane with specialized signaling capacity and recent work demonstrates that TrkB receptors are recruited into lipid rafts upon activation with BDNF (Suzuki et al., 2004). Since SFKs are residents of lipid rafts (by virtue of their lipid modification by myristate) we wondered whether Trk and A2A receptors were found in the same membrane compartments and if they physically associate. The yield of detergent insoluble membrane fractions (i.e., lipid rafts) is generally low, and therefore to be certain we would have enough material for analysis, in these studies we used homogenates of neonatal spinal cord tissue. After homogenization in cold 0.5% TritonX-100, centrifugation through a discontinuous sucrose gradient, and fraction collection, we recovered lipid raft marker Thy-1 immunoreactivity within fraction 2 (by convention “lipid rafts”). Trk receptors, adenosine A2a receptors and active SFK were found in both lipid raft and non-lipid raft fractions (FIG. 6a). Using the clone 28 antibody that recognizes 4 active SFK members (src, fyn, yes and fgr) that differ in their migration through SDS-PAGE gel, we found a predominance of the slower migrating SFK(s) in the lipid raft fraction and a predominance of the faster migrating SFK(s) in the non-lipid raft fractions. Co-immunoprecipitation analysis using beads coated with anti-Trk or anti-phosphoTrk, demonstrated that adenosine A2a receptors associated with Trk receptors in both lipid raft and non-lipid raft fractions. Since we loaded the same amount of protein in each lane of the western blot, and the bands were more intense in the lipid raft fractions, it is likely the association of these proteins is enriched in this subcellular fraction. Co-immunoprecipitation analysis using beads coated with anti-adenosine A2a or anti-phosphoTrk receptors brought down SFK members in the lipid raft and non-lipid raft fractions. In both immunoprecipitates, it was the slower migrating SFK member that associated with adenosine A2a or phospho-Trk receptors in the lipid raft fraction and the faster migrating SFK member associated with adenosine A2a or phospho-Trk receptors in the non-lipid raft fraction (FIG. 6a). These results indicate that in vivo SFKs are part of a physical complex with adenosine A2a receptors and active Trk receptors, who themselves are physically associated. Whether a hetero-trimeric complex exists (active Trk receptors+active SFKs+adenosine A2a receptors) or multiple binary complexes is not known. At the very least, a series of binary complexes exist and those in the lipid raft fraction that include SFKs are molecularly distinct from those in the non-lipid raft fraction. A model of the relationship between adenosine A2a receptors, SFKs and Trk receptors is shown in FIG. 7.

Finally to begin to determine the biological relevance of the lipid raft signaling complex, we asked if their disruption with cholesterol depleting agents would affect the capacity of BDNF to confer excitotoxic sensitivity upon motor neurons. Pure motor neurons were grown in the presence of CT-1 but in the absence of BDNF for 24 hours at which point the cholesterol depleting agent, β-methylcyclodextin (βMCD) or vehicle was added to the cultures. Thirty minutes later, BDNF or vehicle was added to the culture and 3 hours later the excitoticity assay was performed (FIG. 6b). We determined the amount of cholesterol in our pure motor neuron cultures after 30 minutes of βMCD treatment and found an ˜25% reduction in comparison with vehicle treatment (βMCD v. vehicle; 404±31 μg/μg protein versus 550±16 μg/μg protein, n=5 samples, t-test, p<0.001). In terms of cell death, a statistically significant difference between experimental groups was identified by ANOVA (F_{(5,42)}=32.797; P<0.001) and a post hoc analysis (Scheffé with level of significance set at P<0.05) indicated excitotoxic death only occurred in the (+)BDNF/(−)βMCD treatment group (# of motor neurons exposed to vehicle or kainate; 57±7 versus 40±4). No excitotoxic motor neuron death occurred in the (−)BDNF/(+)βMCD treatment group (# of motor neurons exposed to vehicle or kainate; 60±3 versus 60±5) or in the (+)BDNF/(+)βMCD treatment group (# of motor neurons exposed to vehicle or kainate; 58±4 versus 51±5). These results suggest that disruption of lipid rafts by cholesterol depletion interferes with the ability of BDNF signaling to evoke excitotoxic sensitivity.

### Discussion

One approach to developing effective therapy for ALS aims to identify the primary pathophysiological process(es) that initiate cell dysfunction and ultimately death. Despite intensive effort and enormous progress, this remains an elusive goal. Another approach focuses on the identification of physiological processes that render motor neurons vulnerable to insult. Adopting this strategy we have found that the activation of TrkB by BDNF induces a state of susceptibility within motor neurons to insult. Here we show that blocking TrkB activation by a variety of means prevents excitotoxic motor neuron death as well as death caused by the expression of ALS-causing mutant proteins. This unexpectedly broad neuroprotective action of TrkB antagonism drives the inquiry into the various ways cells activate TrkB and regulate downstream signaling cascades.

Many RTKs undergo transactivation by agonists of GPCRs (Daub et al., 1997; Lee and Chao, 2001; Oak et al., 2001; Lee et al., 2002b; Fischer et al., 2004; Zahradka et al., 2004) and our observations that antagonism of adenosine A2a receptors in spinal cord cultures leads to a reduction in TrkB activation complement these results. They imply that under physiological conditions, adenosine A2a and TrkB receptors endogenously interact and active adenosine A2a receptors contribute to the basal level of TrkB activation. Work from the Chao lab shows that TrkA receptor transactivation involves transcription/translation and is restricted to intracellular membranes (probably the Golgi apparatus) (Rajagopal et al., 2004). We identify an additional niche for the TrkB and adenosine receptor interaction: at the plasma membrane concentrated in hot spots. In addition we show that adenosine A2a and TrkB receptor physically interact (either directly or as part of a larger multiprotein complex)—an observation, to our knowledge, not previously reported. The co-localization of the receptors might allow for dynamic, local changes in TrkB activation as a function of adenosinergic neurotransmission. The present work indicates that the reduction in TrkB activation that follows from adenosine A2a blockade underlies, at least in part, its neuroprotective properties. We do not establish that this is the exclusive molecular mechanism and other changes in cellular neurochemistry evoked by adenosine A2a blockade have the potential to be healthful for motor neurons.

SFKs participate in GPCR mediated transactivation of several RTKs (Keely et al., 2000; Lee and Chao, 2001; Krieg et al., 2002; Zahradka et al., 2004)). While intermediates in the pathway, we do not know whether SFKs directly phosphorylate these receptor tyrosine kinases or if the effect is indirect. Similarly, while it is parsimonious to posit that the neuroprotective action of blocking SFKs is due to their ability to decrease TrkB activation, the plethora of SFK substrates (in particular MAPK) raise other possibilities (Murray et al., 1998; Runden et al., 1998; Grewal et al., 1999; Ishikawa et al., 2000; Kaplan and Miller, 2000; Kim et al., 2003; Bromann et al., 2004; Choi et al., 2004; Luttrell and Luttrell, 2004). Regardless of its precise mechanism of action, increases in SFK activity can adversely affect neuronal health (or exacerbate toxic insult, i.e., Aβ peptides) and inhibition of SFK activity can protect neurons from insult (Lambert et al., 1998; Chin et al., 2004; Lennmyr et al., 2004; Chin et al., 2005).

We, and others, have detected RTKs, transactivating GPCRs and SFKs in lipid rafts and their co-localization bolsters the case for the physiological relevance of this membrane compartment as a platform for signal transduction integration (FIG. 7) (Ushio-Fukai et al., 2001; Hur et al., 2004). These specialized membrane domains are dynamic: proteins enter into and egress from lipid rafts upon ligand binding and lipid rafts themselves may associate to bring new signaling capacities to liganded receptors (Simons and Toomre, 2000; Tansey et al., 2000; Paratcha and Ibanez, 2002; Ma et al., 2003; Golub et al., 2004). For example, in cortical neuron cultures, BDNF can recruit a subpopulation of TrkB receptors into lipid rafts and while BDNF-stimulated TrkB receptors preferentially activate MAP kinase in lipid rafts, they activate Akt in non-lipid raft membrane (Suzuki et al., 2004). A subtype of lipid rafts are caveolae—flask-shaped membrane invaginations that contain the cholesterol-binding protein caveolin as well as a diversity of receptors and signaling molecules (Krajewska and Maslowska, 2004). Dissolution of lipid rafts by cholesterol depletion can block transactivation of the epidermal growth factor RTK by the angiotensin II GPCR AT₁, in a process that involves caveolin (Ushio-Fukai et al., 2001). These findings might be relevant to our current observations (i.e., FIG. 6) since Trk receptors have been shown to reside in caveolae (at least in PC12 cells) and physically associate with caveolin (Bilderback et al., 1999; Peiro et al., 2000). Future work will be needed to explore the differences in signals propagating from TrkB receptors activated by BDNF versus those transactivated by adenosine A2a receptors as a function of lipid raft integrity.

For two reasons we believe the neuroprotective activity of adenosine A2a receptor antagonism is operating in motor neurons themselves. First, motor neurons express both A2a receptors and TrkB receptors and they are co-localized into distinct subcellular domains (punta) suggesting a privileged signaling capacity. Second, A2a receptor antagonists protect purified motor neurons from insult indicating the lack of necessity of non-motor neurons and glial cells in the biological effect. Thus while ALS is increasingly being viewed a disorder that involves the participation of multiple cell types (i.e. astrocytes, muscle, microglia), the present results highlight motor neuron-specific processes (Clement et al., 2003; Dupuis et al., 2003; Barbeito et al., 2004; Dupuis et al., 2004; Pehar et al., 2004; Cassina et al., 2005; Schutz et al., 2005).

Several investigators have found that a pathological rise in intracellular calcium is an essential component of the mechanism of excitotoxic insult to motor neurons (Carriedo et al., 1996) and interestingly, the toxicity of mutant forms of SOD in vitro are abrogated by maneuvers that antagonize AMPA receptor activation and limit rises in intracellular calcium (Roy et al., 1998). Since activation of Trk receptors or adenosine A2a receptors can modulate intracellular calcium levels, our current studies in mixed cell cultures might reflect effects on modulation of intracellular calcium levels, particularly in specific sub-cellular domains. In a variety of neuronal preparations, application of BDNF leads to a rise in intracellular calcium in a tyrosine kinase-dependent manner (Mizoguchi et al., 2002; Lamb and Bielefeldt, 2003; Mizoguchi and Nabekura, 2003). With regard to adenosine A2a receptors, the situation is more complex; their blockade on pre-synaptic terminals prevents stimulus-evoked rises in intra-terminal calcium levels (Correia-de-Sa et al., 2000; Li and Wong, 2000) while their activation on the cell soma blocks hypoxia-induced rises in intracellular calcium (Kobayashi et al., 1998). Given these observations, reduction in TrkB activation on the cell soma or adenosine A2a receptors on pre-synaptic terminals might have neuroprotective effects by blunting rises in intracellular calcium that follow noxious insult.

Previously we showed that agonist-evoked rises in intracellular calcium are not higher in purified motor neurons grown in the presence of BDNF versus those grown in the presence of other trophic factors (Fryer et al., 2000). Thus, while synaptic activity can be enhanced by BDNF-TrkB signaling (Poo, 2001), it is unlikely that BDNF makes motor neurons vulnerable to excitotoxic insult simply by potentiating the capacity of glutamate to excite neurons. Our results indicate that activity-dependent rise in intracellular calcium is necessary (but not sufficient) for motor neuron death (Fryer et al., 1999) and that the effects of BDNF, in our experimental paradigms, are downstream of this rise in intracellular calcium.

One of the particular attractions of the present observations is the potential ability to translate them into human therapeutics. Trk antagonists related to CEP4416 (Cephalon, Inc.) are in clinical use currently for the treatment of leukemia and solid tumors (Smith et al., 2004; Undevia et al., 2004). After peripheral administration, CEP4416 is detectable in central nervous system tissues and causes a reduction in the abundance of activated Trk (not shown). The long-term utility of Trk antagonists may be limited by their effects on the maintenance of neuromuscular junction integrity (Gonzalez et al., 1999) and learning and memory (Korte et al., 1996; Messaoudi et al., 2002; Pang et al., 2004). Adenosine A2a receptor antagonists have been shown to be neuroprotective agents in animal models of parkinsonism (Shiozaki et al., 1999; Koga et al., 2000; Ikeda et al., 2002; Fink et al., 2004). Potential adverse effects on inflammation (Ohta and Sitkovsky, 2001; Thiel et al., 2005) and coronary artery vaso-regulation (Belardinelli et al., 1998) have not limited their safe use in humans with Parkinson's Disease (Bara-Jimenez et al., 2003; Kase et al., 2003). Seven transmembrane domain receptors (such as the adenosine A2a receptors) are the most common target of therapeutic drugs (Lefkowitz and Shenoy, 2005), raising the possibility that future agents may be developed that antagonize the pathway by which A2a receptors transactivate Trk receptors. Although in vitro observations on ALS therapeutics do not invariably translate into in vivo efficacy (cf. (Gurney et al., 1996; Li et al., 2000) versus (Groeneveld et al., 2003)), this combination of favorable characteristic could hasten the evaluation of adenosine A2a or Trk antagonists for the treatment of ALS.

### REFERENCES

Bara-Jimenez W, Sherzai A, Dimitrova T, Favit A, Bibbiani F, Gillespie M, Morris M J, Mouradian M M, Chase T N (2003) Adenosine A(2A) receptor antagonist treatment of Parkinson's disease. Neurology 61:293-296.
Barbeito L H, Pehar M, Cassina P, Vargas M R, Peluffo H, Viera L, Estevez A G, Beckman J S (2004) A role for astrocytes in motor neuron loss in amyotrophic lateral sclerosis. Brain Res Brain Res Rev 47:263-274.
Belardinelli L, Shryock J C, Snowdy S, Zhang Y, Monopoli A, Lozza G, Ongini E, Olsson R A, Dennis D M (1998) The A2A adenosine receptor mediates coronary vasodilation. J Pharmacol Exp Ther 284:1066-1073.
Bilderback T R, Gazula V R, Lisanti M P, Dobrowsky R T (1999) Caveolin interacts with Trk A and p75(NTR) and regulates neurotrophin signaling pathways. J Biol Chem 274:257-263.
Boulanger L, Poo M (1999) Gating of BDNF-induced synaptic potentiation by cAMP. Science 284:1982-1984.
Bromann P A, Korkaya H, Courtneidge S A (2004) The interplay between Src family kinases and receptor tyrosine kinases. Oncogene 23:7957-7968.
Bruijn L I, Miller T M, Cleveland D W (2004) Unraveling the mechanisms involved in motor neuron degeneration in ALS. Annu Rev Neurosci 27:723-749.
Carriedo S G, Yin H Z, Weiss J H (1996) Motor neurons are selectively vulnerable to AMPA/KA receptor-mediated injury in vitro. J Neurosci 16:4069-4079.
Carriedo S G, Sensi S L, Yin H Z, Weiss J H (2000) AMPA exposures induce mitochondrial Ca(2+) overload and ROS generation in spinal motor neurons in vitro. J Neurosci 20:240-250.
Cassina P, Pehar M, Vargas M R, Castellanos R, Barbeito A G, Estevez A G, Thompson J A, Beckman J S, Barbeito L (2005) Astrocyte activation by fibroblast growth factor-1 and motor neuron apoptosis: implications for amyotrophic lateral sclerosis. J Neurochem 93:38-46.
Chin J, Palop J J, Yu G Q, Kojima N, Masliah E, Mucke L (2004) Fyn kinase modulates synaptotoxicity, but not aberrant sprouting, in human amyloid precursor protein transgenic mice. J Neurosci 24:4692-4697.
Chin J, Palop J J, Puolivali J, Massaro C, Bien-Ly N, Gerstein H, Scearce-Levie K, Masliah E, Mucke L (2005) Fyn kinase induces synaptic and cognitive impairments in a transgenic mouse model of Alzheimer's disease. J Neurosci 25:9694-9703.
Choi S Y, Hwang J J, Koh J Y (2004) NR2A induction and NMDA receptor-dependent neuronal death by neurotrophin-4/5 in cortical cell culture. J Neurochem 88:708-716.
Clement A M, Nguyen M D, Roberts E A, Garcia M L, Boillee S, Rule M, McMahon A P, Doucette W, Siwek D, Ferrante R J, Brown R H, Jr., Julien J P, Goldstein L S, Cleveland D W (2003) Wild-type nonneuronal cells extend survival of SOD1 mutant motor neurons in ALS mice. Science 302:113-117.
Correia-de-Sa P, Timoteo M A, Ribeiro J A (2000) A(2A) adenosine receptor facilitation of neuromuscular transmission: influence of stimulus paradigm on calcium mobilization. J Neurochem 74:2462-2469.
Cox P A, Banack S A, Murch S J (2003) Biomagnification of cyanobacterial neurotoxins and neurodegenerative disease among the Chamorro people of Guam. Proc Natl Acad Sci U S A 100:13380-13383.
Daub H, Wallasch C, Lankenau A, Herrlich A, Ullrich A (1997) Signal characteristics of G protein-transactivated EGF receptor. Embo J 16:7032-7044.
Downward J (2003) Role of receptor tyrosine kinases in G-protein-coupled receptor regulation of Ras: transactivation or parallel pathways? Biochem J 376:e9-10.
Dunwiddie T V, Masino S A (2001) The role and regulation of adenosine in the central nervous system. Annu Rev Neurosci 24:31-55.
Dunwiddie T V, Diao L, Proctor W R (1997) Adenine nucleotides undergo rapid, quantitative conversion to adenosine in the extracellular space in rat hippocampus. J Neurosci 17:7673-7682.
Dupuis L, Oudart H, Rene F, Gonzalez de Aguilar J L, Loeffler J P (2004) Evidence for defective energy homeostasis in amyotrophic lateral sclerosis: benefit of a high-energy diet in a transgenic mouse model. Proc Natl Acad Sci USA 101:11159-11164.
Dupuis L, di Scala F, Rene F, de Tapia M, Oudart H, Pradat P F, Meininger V, Loeffler J P (2003) Up-regulation of mitochondrial uncoupling protein 3 reveals an early muscular metabolic defect in amyotrophic lateral sclerosis. Faseb J 17:2091-2093.
Eguchi S, Dempsey P J, Frank G D, Motley E D, Inagami T (2001) Activation of MAPKs by angiotensin II in vascular smooth muscle cells. Metalloprotease-dependent EGF receptor activation is required for activation of ERK and p38 MAPK but not for JNK. J Biol Chem 276:7957-7962.
Emfors P, Lee K, F., Jaenisch R (1995) Mice lacking brain-derived neurotrophic factor develop with sensory defi its. Nature 368:147-150.
Estevez A G, Stutzmann J M, Barbeito L (1995) Protective effect of riluzole on excitatory amino acid-mediated neurotoxicity in motoneuron-enriched cultures. Eur J Pharmacol 280:47-53.
Evans A E, Kisselbach K D, Yamashiro D J, Ikegaki N, Camoratto A M, Dionne C A, Brodeur G M (1999) Antitumor activity of CEP-751 (KT-6587) on human neuroblastoma and medulloblastoma xenografts. Clin Cancer Res 5:3594-3602.
Evans A E, Kisselbach K D, Liu X, Eggert A, Ikegaki N, Camoratto A M, Dionne C, Brodeur G M (2001) Effect of CEP-751 (KT-6587) on neuroblastoma xenografts expressing TrkB. Med Pediatr Oncol 36:181-184.
Fabrizio P, Pozza F, Pletcher S D, Gendron C M, Longo V D (2001) Regulation of longevity and stress resistance by Sch9 in yeast. Science 292:288-290.
Fink J S, Kalda A, Ryu H, Stack E C, Schwarzschild M A, Chen J F, Ferrante R J (2004) Genetic and pharmacological inactivation of the adenosine A2A receptor attenuates 3-nitropropionic acid-induced striatal damage. J Neurochem 88:538-544.
Fischer O M, Giordano S, Comoglio P M, Ullrich A (2004) Reactive oxygen species mediate Met receptor transactivation by G protein-coupled receptors and the epidermal growth factor receptor in human carcinoma cells. J Biol Chem 279:28970-28978.
Fredholm B B, APU, Jacobson K A, Klotz K N, Linden J (2001) International Union of Pharmacology. XXV. Nomenclature and classification of adenosine receptors. Pharmacol Rev 53:527-552.
Fryer H J L, Knox R J, Strittmatter S M, Kalb R G (1999) Excitotoxic death of a subset of embryonic rat motor neurons in vitro. J Neurochem 72:500-513.
Fryer H J L, Wolf D H, Knox R J, Strittmatter S M, Pennica D, O'Leary R M, Russell D S, Kalb R G (2000) Brain-derived neurotrophic factor induces excitotoxic sensitivity in cultured embryonic rat spinal motor neurons through activation of the phosphatidylinositol 3-kinase pathway. J Neurochem 74:582-595.
Golub T, Wacha S, Caroni P (2004) Spatial and temporal control of signaling through lipid rafts. Curr Opin Neurobiol 14:542-550.
Gonzalez M, Ruggiero F P, Chang Q, Shi Y-J, Rich M M, Kraner S, Balice-Gordon R J (1999) Disruption of TrkB-mediated signaling induces disassembly of postsynaptic receptor clusters at the neuromuscular junction. Neuron 24:567-583.
Grewal S S, York R D, Stork P J (1999) Extracellular-signal-regulated kinase signalling in neurons. Curr Opin Neurobiol 9:544-553.
Groeneveld G J, Veldink J H, van der Tweel I, Kalmijn S, Beijer C, de Visser M, Wokke J H, Franssen H, van den Berg L H (2003) A randomized sequential trial of creatine in amyotrophic lateral sclerosis. Ann Neurol 53:437-445.
Gurney M E, Cutting F B, Zhai P, Doble A, Taylor C P, Andrus P K, Hall E D (1996) Benefit of vitamin E, riluzole, and gabapentin in a transgenic model of familial amyotrophic lateral sclerosis. Ann Neurol 39:147-157.
Hanke J H, Gardner J P, Dow R L, Changelian P S, Brissette W H, Weringer E J, Pollok B A, Connelly P A (1996) Discovery of a novel, potent, and Src family-selective tyrosine kinase inhibitor. Study of Lck- and FynT-dependent T cell activation. J Biol Chem 271:695-701.
Hanson M G, Jr., Shen S, Wiemelt A P, McMorris F A, Barres B A (1998) Cyclic A M P elevation is sufficient to promote the survival of spinal motor neurons in vitro. J Neurosci 18:7361-7371.
Henderson C E, Camu W, Mettling C, Gouin A, Poulsen K, Karihaloo M, Rullamas J, Evans T, McMahon S B, Armanini M P, Berkemeier L, Phillips H S, Rosenthal A (1993) Neurotrophins promote motor neuron survival and are present in embryonic limb bud. Nature 363:266-270.
Hirschhorn R, Roegner-Maniscalco V, Kuritsky L, Rosen F S (1981) Bone marrow transplantation only partially restores purine metabolites to normal in adenosine deaminase-deficient patients. J Clin Invest 68:1387-1393.
Hu P, Kalb R G (2003) BDNF heightens the sensitivity of motor neurons to excitotoxic insults through activation of TrkB. J Neurochem 84:1421-1430.
Hur E M, Park Y S, Lee B D, Jang I H, Kim H S, Kim T D, Suh P G, Ryu S H, Kim K T (2004) Sensitization of epidermal growth factor-induced signaling by bradykinin is mediated by c-Src. Implications for a role of lipid microdomains. J Biol Chem 279:5852-5860.
Ikeda K, Kurokawa M, Aoyama S, Kuwana Y (2002) Neuroprotection by adenosine A2A receptor blockade in experimental models of Parkinson's disease. J Neurochem 80:262-270.
Ishikawa Y, Ikeuchi T, Hatanaka H (2000) Brain-derived neurotrophic factor accelerates nitric oxide donor-induced apoptosis of cultured cortical neurons. J Neurochem 75:494-502.
Ji Y, Pang P T, Feng L, Lu B (2005) Cyclic AMP controls BDNF-induced TrkB phosphorylation and dendritic spine formation in mature hippocampal neurons. Nat Neurosci 8:164-172.
Jones K, R., Farinas I, Backus C, Reichardt L, F (1994) Targeted Disruption of the BDNF Gene Perturbs Brain and Sensory Neuron Development but not Motor Neuron Development. Cell 76:989-999.
Kalb R (2005) The protean actions of neurotrophins and their receptors on the life and death of neurons. Trends Neurosci 28:5-11.
Kaplan D R, Miller F D (2000) Neurotrophin signal transduction in the nervous system. Curr Opin Neurobiol 10:381-391.
Kase H, Aoyama S, Ichimura M, Ikeda K, Ishii A, Kanda T, Koga K, Koike N, Kurokawa M, Kuwana Y, Mori A, Nakamura J, Nonaka H, Ochi M, Saki M, Shimada J, Shindou T, Shiozaki S, Suzuki F, Takeda M, Yanagawa K, Richardson P J, Jenner P, Bedard P, Borrelli E, Hauser R A, Chase T N (2003) Progress in pursuit of therapeutic A2A antagonists: the adenosine A2A receptor selective antagonist KW6002: research and development toward a novel nondopaminergic therapy for Parkinson's disease. Neurology 61:S97-100.
Kawabuchi M, Satomi Y, Takao T, Shimonishi Y, Nada S, Nagai K, Tarakhovsky A, Okada M (2000) Transmembrane phosphoprotein Cbp regulates the activities of Src-family tyrosine kinases. Nature 404:999-1003.
Kawakatsu H, Sakai T, Takagaki Y, Shinoda Y, Saito M, Owada M K, Yano J (1996) A new monoclonal antibody which selectively recognizes the active form of Src tyrosine kinase. J Biol Chem 271:5680-5685.
Keely S J, Calandrella S O, Barrett K E (2000) Carbachol-stimulated transactivation of epidermal growth factor receptor and mitogen-activated protein kinase in T(84) cells is mediated by intracellular ca(2+), PYK-2, and p60(src). J Biol Chem 275:12619-12625.
Kim H J, Hwang J J, Behrens M M, Snider B J, Choi D W, Koh J Y (2003) TrkB mediates BDNF-induced potentiation of neuronal necrosis in cortical culture. Neurobiol Dis 14:110-119.
Kobayashi S, Conforti L, Pun R Y, Millhom D E (1998) Adenosine modulates hypoxia-induced responses in rat PC12 cells via the A2A receptor. J Physiol 508 (Pt 1):95-107.
Koga K, Kurokawa M, Ochi M, Nakamura J, Kuwana Y (2000) Adenosine A(2A) receptor antagonists KFI7837 and KW-6002 potentiate rotation induced by dopaminergic drugs in hemi-Parkinsonian rats. Eur J Pharmacol 408:249-255.
Koh J Y, Gwag B J, Lobner D, Choi D W (1995) Potentiated necrosis of cultured cortical neurons by neurotrophins. Science 268:573-575.
Korte M, Staiger V, Griesbeck O, Thoenen H, Bonhoeffer T (1996) The involvement of brain-derived neurotrophic factor in hippocampal long-term potentiation revealed by gene targeting experiments. J Physiol Paris 90:157-164.
Krajewska W M, Maslowska 1 (2004) Caveolins: structure and function in signal transduction. Cell Mol Biol Lett 9:195-220.
Krieg T, Qin Q, McIntosh E C, Cohen M V, Downey J M (2002) ACh and adenosine activate P13-kinase in rabbit hearts through transactivation of receptor tyrosine kinases. Am J Physiol Heart Circ Physiol 283:H2322-2330.
Kruman, I I, Pedersen W A, Springer J E, Mattson M P (1999) ALS-linked Cu/Zn-SOD mutation increases vulnerability of motor neurons to excitotoxicity by a mechanism involving increased oxidative stress and perturbed calcium homeostasis. Exp Neurol 160:28-39.
Kust B M, Copray J C, Brouwer N, Troost D, Boddeke H W (2002) Elevated levels of neurotrophins in human biceps brachii tissue of amyotrophic lateral sclerosis. Exp Neurol 177:419-427.
Kwak S, Nakamura R (1995) Acute and late neurotoxicity in the rat spinal cord in vivo induced by glutamate receptor agonists. J Neurol Sci 129 Suppl:99-103.
Lamb K, Bielefeldt K (2003) Rapid effects of neurotrophic factors on calcium homeostasis in rat visceral afferent neurons. Neurosci Lett 336:9-12.
Lambert M P, Barlow A K, Chromy B A, Edwards C, Freed R, Liosatos M, Morgan T E, Rozovsky I, Trommer B, Viola K L, Wals P, Zhang C, Finch C E, Krafft G A, Klein W L (1998) Diffusible, nonfibrillar ligands derived from Abetal-42 are potent central nervous system neurotoxins. Proc Natl Acad Sci USA 95:6448-6453.
Lee F S, Chao M V (2001) Activation of Trk neurotrophin receptors in the absence of neurotrophins. Proc Natl Acad Sci USA 98:3555-3560.
Lee F S, Rajagopal R, Chao M V (2002a) Distinctive features of Trk neurotrophin receptor transactivation by G protein-coupled receptors. Cytokine Growth Factor Rev 13:11-17.
Lee F S, Rajagopal R, Kim A H, Chang P C, Chao M V (2002b) Activation of Trk neurotrophin receptor signaling by pituitary adenylate cyclase-activating polypeptides. J Biol Chem 277:9096-9102.
Lefkowitz R J, Shenoy S K (2005) Transduction of receptor signals by beta-arrestins. Science 308:512-517.
Lemmyr F, Ericsson A, Gerwins P, Akterin S, Ahlstrom H, Terent A (2004) Src family kinase-inhibitor PP2 reduces focal ischemic brain injury. Acta Neurol Scand 110: 175-179.
Li M, Ona V O, Guegan C, Chen M, Jackson-Lewis V, Andrews L J, Olszewski A J, Stieg P E, Lee J P, Przedborski S, Friedlander R M (2000) Functional role of caspase-1 and caspase-3 in an ALS transgenic mouse model. Science 288:335-339.
Li S N, Wong P T (2000) The adenosine receptor agonist, APNEA, increases calcium influx into rat cortical synaptosomes through N-type channels associated with A2a receptors. Neurochem Res 25:457-459.
Liu X, Emfors P, Wu H, Jaenisch R (1995) Sensory but not motor neuron deficits in mice lacking NT4 and BDNF. Nature 375:238-241.
Luttrell D K, Luttrell L M (2004) Not so strange bedfellows: G-protein-coupled receptors and Src family kinases. Oncogene 23:7969-7978.
Luttrell L M, Daaka Y, Lefkowitz R J (1999) Regulation of tyrosine kinase cascades by G-protein-coupled receptors. Curr Opin Cell Biol 11:177-183.
Ma L, Huang Y Z, Pitcher G M, Valtschanoff J G, Ma Y H, Feng L Y, Lu B, Xiong W C, Salter M W, Weinberg R J, Mei L (2003) Ligand-dependent recruitment of the ErbB4 signaling complex into neuronal lipid rafts. J Neurosci 23:3164-3175.
Messaoudi E, Ying S W, Kanhema T, Croll S D, Bramham C R (2002) Brain-derived neurotrophic factor triggers transcription-dependent, late phase long-term potentiation in vivo. J Neurosci 22:7453-7461.
Mizoguchi Y, Nabekura J (2003) Sustained intracellular Ca2+ elevation induced by a brief BDNF application in rat visual cortex neurons. Neuroreport 14:1481-1483.
Mizoguchi Y, Monji A, Nabekura J (2002) Brain-derived neurotrophic factor induces long-lasting Ca2+-activated K⁺ currents in rat visual cortex neurons. Eur J Neurosci 16:1417-1424.
Mojsilovic-Petrovic J, Arneja A, Kalb R (2005) Enprofylline protects motor neurons from in vitro excitotoxic challenge. Neurodegenerative Diseases 2:160-165.
Mukhopadhyay D, Tsiokas L, Zhou X M, Foster D, Brugge J S, Sukhatme V P (1995) Hypoxic induction of human vascular endothelial growth factor expression through c-Src activation. Nature 375:577-581.
Murray B, Alessandrini A, Cole A J, Yee A G, Furshpan E J (1998) Inhibition of the p44/42 MAP kinase pathway protects hippocampal neurons in a cell-culture model of seizure activity. Proc Natl Acad Sci USA 95:11975-11980.
Neve R L, Howe J R, Hong S, Kalb R G (1997) Introduction of the glutamate receptor subunit 1 into motor neurons in vitro and in vivo using a recombinant herpes simplex virus. Neuroscience 79:435-447.
Oak J N, Lavine N, Van Tol H H (2001) Dopamine D(4) and D(2L) Receptor Stimulation of the Mitogen-Activated Protein Kinase Pathway Is Dependent on trans-Activation of the Platelet-Derived Growth Factor Receptor. Mol Pharmacol 60:92-103.
Ohta A, Sitkovsky M (2001) Role of G-protein-coupled adenosine receptors in downregulation of inflammation and protection from tissue damage. Nature 414:916-920.
Pang P T, Teng H K, Zaitsev E, Woo N T, Sakata K, Zhen S, Teng K K, Yung W H, Hempstead B L, Lu B (2004) Cleavage of proBDNF by tPA/plasmin is essential for long-term hippocampal plasticity. Science 306:487-491.
Paratcha G, Ibanez C F (2002) Lipid rafts and the control of neurotrophic factor signaling in the nervous system: variations on a theme. Curr Opin Neurobiol 12:542-549.
Pehar M, Cassina P, Vargas M R, Castellanos R, Viera L, Beckman J S, Estevez A G, Barbeito L (2004) Astrocytic production of nerve growth factor in motor neuron apoptosis: implications for amyotrophic lateral sclerosis. J Neurochem 89:464-473.
Peiro S, Comella J X, Enrich C, Martin-Zanca D, Rocamora N (2000) PC12 cells have caveolae that contain TrkA. Caveolae-disrupting drugs inhibit nerve growth factor-induced, but not epidermal growth factor-induced, MAPK phosphorylation. J Biol Chem 275:37846-37852.
Piiper A, Zeuzem S (2004) Receptor tyrosine kinases are signaling intermediates of G protein-coupled receptors. Curr Pharm Des 10:3539-3545.
Poo M (2001) Neurotrophins as synaptic modulators. Nat Rev Neurosci 2:24-32.
Puls I, Jonnakuty C, LaMonte B H, Holzbaur E L, Tokito M, Mann E, Floeter M K, Bidus K, Drayna D, Oh S J, Brown R H, Ludlow C L, Fischbeck K H (2003) Mutant dynactin in motor neuron disease. Nat Genet.
Rajagopal R, Chen Z Y, Lee F S, Chao M V (2004) Transactivation of Trk neurotrophin receptors by G-protein-coupled receptor ligands occurs on intracellular membranes. J Neurosci 24:6650-6658.
Robeva S A, Woodard R L, Jin X, Gao Z, Bhattacharya S, Taylor H E, Rosin D L, Linden J (1996) Molelcular characterization of recombinant human adenosine receptors. Drug Development Research 39:243-252.
Rosen DLea (1993) Mutations in Cu/Zn superoxide dismutase gene are associated with familial amyotrophic lateral sclerosis. Nature 362:59-62.
Rothstein J D, Jin L, Dykes-Hoberg M, Kuncl R W (1993) Chronic inhibition of glutamate uptake produces a model of slow neurotoxicity. Proc Natl Acad Sci USA 90:6591-6595.
Rothstein J D, Patel S, Regan M R, Haenggeli C, Huang Y H, Bergles D E, Jin L, Dykes Hoberg M, Vidensky S, Chung D S, Toan S V, Bruijn L I, Su Z Z, Gupta P, Fisher P B (2005) Beta-lactam antibiotics offer neuroprotection by increasing glutamate transporter expression. Nature 433:73-77.
Roy J, Minotti S, Dong L, Figlewicz D A, Durham H D (1998) Glutamate potentiates the toxicity of mutant Cu/Zn-superoxide dismutase in motor neurons by postsynaptic calcium-dependent mechanisms. J Neurosci 18:9673-9684.
Runden E, Seglen P O, Haug F M, Ottersen O P, Wieloch T, Shamloo M, Laake J H (1998) Regional selective neuronal degeneration after protein phosphatase inhibition in hippocampal slice cultures: evidence for a MAP kinase-dependent mechanism. J Neurosci 18:7296-7305.
Schutz B, Reimann J, Dumitrescu-Ozimek L, Kappes-Horn K, Landreth G E, Schurmann B, Zimmer A, Heneka M T (2005) The oral antidiabetic pioglitazone protects from neurodegeneration and amyotrophic lateral sclerosis-like symptoms in superoxide dismutase-G93A transgenic mice. J Neurosci 25:7805-7812.
Schwartz P M, Borghesani P R, Levy R L, Pomeroy S L, Segal R A (1997) Abnormal cerebellar development and foliation in BDNF−/− mice reveals a role for neurotrophins in CNS patterning. Neuron 19:269-281.
Shiozaki S, Ichikawa S, Nakamura J, Kitamura S, Yamada K, Kuwana Y (1999) Actions of adenosine A2A receptor antagonist KW-6002 on drug-induced catalepsy and hypokinesia caused by reserpine or MPTP. Psychopharmacology (Ber1) 147:90-95.
Silos-Santiago I, Fagan A M, Garber M, Fritzsch B, Barbacid M (1997) Severe sensory deficits but normal CNS development in newborn mice lacking TrkB and TrkC tyrosine protein kinase receptors. Eur J Neurosci 9:2045-2056.
Simons K, Toomre D (2000) Lipid rafts and signal transduction. Nat Rev Mol Cell Biol 1:31-39.
Smith B D, Levis M, Beran M, Giles F, Kantarjian H, Berg K, Murphy K, Dauses T, Allebach J, Small D (2004) Single agent CEP-701, a novel FLT3 inhibitor, shows biological and clinical activity in patients with relapsed or refractory acute myeloid leukemia. Blood Jan 15 (Epub ahead of print).
Suzuki S, Numakawa T, Shimazu K, Koshimizu H, Hara T, Hatanaka H, Mei L, Lu B, Kojima M (2004) BDNF-induced recruitment of TrkB receptor into neuronal lipid rafts: roles in synaptic modulation. J Cell Biol 167:1205-1215.
Tansey M G, Baloh R H, Milbrandt J, Johnson E M, Jr. (2000) GFRalpha-mediated localization of RET to lipid rafts is required for effective downstream signaling, differentiation, and neuronal survival. Neuron 25:611-623.
Thiel M, Chouker A, Ohta A, Jackson E, Caldwell C, Smith P, Lukashev D, Bittmann I, Sitkovsky M V (2005) Oxygenation inhibits the physiological tissue-protecting mechanism and thereby exacerbates acute inflammatory lung injury. PLoS Biol 3:e174.
Tsvyetlynska N A, Hill R H, Grillner S (2005) Role of AMPA receptor desensitization and the side effects of a DMSO vehicle on reticulospinal EPSPs and locomotor activity. J Neurophysiol 94:3951-3960.
Undevia S D, Vogelzang N J, Mauer A M, Janisch L, Mani S, Ratain M J (2004) Phase I clinical trial of CEP-2563 dihydrochloride, a receptor tyrosine kinase inhibitor, in patients with refractory solid tumors. Invest New Drugs 22:449-458.
Ushio-Fukai M, Hilenski L, Santanam N, Becker P L, Ma Y, Griendling K K, Alexander R W (2001) Cholesterol depletion inhibits epidermal growth factor receptor transactivation by angiotensin II in vascular smooth muscle cells: role of cholesterol-rich microdomains and focal adhesions in angiotensin II signaling. J Biol Chem 276:48269-48275.
Van Damme P, Leyssen M, Callewaert G, Robberecht W, Van Den Bosch L (2003) The AMPA receptor antagonist NBQX prolongs survival in a transgenic mouse model of amyotrophic lateral sclerosis. Neurosci Lett 343:81-84.
Waters C, Pyne S, Pyne N J (2004) The role of G-protein coupled receptors and associated proteins in receptor tyrosine kinase signal transduction. Semin Cell Dev Biol 15:309-323.
Zahradka P, Litchie B, Storie B, Helwer G (2004) Transactivation of the insulin-like growth factor-I receptor by angiotensin II mediates downstream signaling from the angiotensin II type 1 receptor to phosphatidylinositol 3-kinase. Endocrinology 145:2978-2987.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for inhibiting neurodegeneration associated with TrkB signaling, comprising administration of an effective amount of an A2a adenosine receptor antagonist to a patient in need thereof.

2. The method of claim 1, wherein said antagonist is selected from the group consisting of enprofylline, ZM241385, KW6002, KF17837, 1,3,7-trimethyl-8-styrylxanthine, 1,3,7-trimethyl-8-(2-methoxystyryl)xanthine, 1,3,7-trimethyl-8-(3-methoxystyryl)xanthine, 1,3,7-trimethyl-8-[3-(trifluoromethyl)styryl]xanthine, 1,3,7-trimethyl-8-(3-nitrostyryl)xanthine, 1,3,7-trimethyl-8-(3-aminostyryl)xanthine, 1,3,7-trimethyl-8-[3-(acetylamino)styryl]xanthine, 1,3,7-trimethyl-8-[3-[3(-carboxyl-1-oxopropyl)amino]-styryl]xanthine, 1,3,7-trimethyl-8-[3-[(tert-butyloxy)carbonyl]amino]-styryl]xanthine, 1,3,7-trimethyl-8-[3-[bis[(tert-butyloxy)carbonyl]amino]-styryl]xanthine, 1,3,7-trimethyl-8-(3-fluorostyryl)xanthine, 1,3,7-trimethyl-8-(4-methoxystyryl)xanthine, 1,3,7-trimethyl-8-(3,4-dimethoxystyryl)xanthine, 1,3-dimethyl-8-(3,5-dimethoxystyryl)xanthine, 1,3,7-trimethyl-8-(3,5-dimethoxystyryl)xanthine, 1,3,7-trimethyl-8-(3,5-difluorostyryl)xanthine, 1,3,7-trimethyl-8-[3,5-dimethoxy-4-(hydroxy)styryl]xanthine, 1,3,7-trimethyl-8-[3,5-dimethoxy-4-(acetoxy)styryl]xanthine, 1,3,7-trimethyl-8-[3,5-dimethoxy-4-(benzyloxy)styryl]xanthine, 1,3,7-trimethyl-8-[3,5-dimethoxy-4->(4-aminobutyl)oxy]-styryl]xanthine, 1,3,7-trimethyl-8-[3,5-dimethoxy-4-[[4-[[(tertbutyloxy)-carbonyl]amino]butyl]oxy]styryl]xanthine, 1,3,7,-trimethyl-8-[3,5-dimethoxy-4-[(4-amino-trans-butenyl)oxy]styryl]xanthine, 1,3,7-trimethyl-8-[3,5-dimethoxy-4-[(4-acetylamino-trans-butenyl)oxy]styryl]xanthine, 1,3,7-trimethyl-8-[3,5-dimethoxy-4-[(4-t-butyloxycarbonylamino-trans-butenyl)oxy]styryl]xanthine, 1,3,7-trimethyl-8-(2,3,4-trimethoxystyryl)xanthine, 1,3,7-trimethyl-8-(3,4,5-trimethoxystyryl)xanthine, 7-Methyl-1,3-diethyl-8-(3,4,5-trimethoxystyryl)xanthine, 7-Methyl-1,3-diallyl-8-(3,4,5-trimethoxystyryl)xanthine, 1,3-dipropyl-7-methyl-8-(3-chlorostyryl)xanthine, 1,3-dipropyl-7-methyl-8-(3,4-dimethoxystyryl)xanthine, 1,3-dipropyl-7-methyl-8-(3,5-dimethoxystyryl)xanthine, DMPX, and SCH58261.

3. The method of claim 1, further comprising administration of a TRK-B receptor antagonist.

4. The method of claim 4, wherein said TRK-B antagonist is CEP4416.

5. The method of claim 1, wherein said agent is administered via a route selected from the group consisting of systemic administration, parenteral administration, intravenous administration, and intracerebral infusion.

6. The method of claim 1, wherein said patient has amyotrophic lateral sclerosis (ALS).

7. A method for identifying agents which protect neurons from toxic insult, comprising:
a) providing a culture of neuronal cells and exposing said cells to excitotoxic conditions which promote neuron cell death;
b) incubating said cells in the presence and absence of said agent; and
c) determining whether said agent exerts a protective effect on said neurons.

8. The method as claimed in claim 7, wherein said agent inhibits neuron cell death.

9. The method of claim 7, wherein said agent inhibits phosphorylation of TrkB and SFK.

10. The method of claim 7, wherein said agent prevents TRK-B receptor signaling, with the proviso said agent is not an antibody having affinity for TRK-B.

11. The method of claim 7, wherein said agent is an adenosine A2a receptor antagonist.

12. The method of claim 7 wherein said agent disrupts the interaction between TRK-B and adenosine A2a receptor.
